SEQUENCE LISTING (250 sequences)

SEQ ID NO: 1 - DNA, 12 bp, Artificial Sequence
  Description of Artificial Sequence Synthetic primer
agcctgcgtggc

SEQ ID NO: 2 - DNA, 18 bp, Artificial Sequence
  Description of Artificial Sequence Synthetic primer
ncggacacccaaagtagt

SEQ ID NO: 3 - DNA, 20 bp, Artificial Sequence
  Description of Artificial Sequence Synthetic primer
cggcycctgaatgyggctaa

SEQ ID NO: 4 - DNA, 13 bp, Artificial Sequence
  Description of Artificial Sequence Synthetic primer
ggctgcgytggcg

SEQ ID NO: 5 - DNA, 18 bp, Artificial Sequence
  Description of Artificial Sequence Synthetic primer
ncggacacccaaagtagt

SEQ ID NO: 6 - DNA, 27 bp, Artificial Sequence
  Description of Artificial Sequence Synthetic primer
acawggtgygaagagtytattgagcta

SEQ ID NO: 7 - DNA, 20 bp, Artificial Sequence
  Description of Artificial Sequence Synthetic primer
tgatcaaattgctagtcttg

SEQ ID NO: 8 - DNA, 23 bp, Artificial Sequence
  Description of Artificial Sequence Synthetic primer
ncttattcaaaattttctgtctg

SEQ ID NO: 9 - DNA, 21 bp, Artificial Sequence
  Description of Artificial Sequence Synthetic primer
aggatgccactaaacctcagc

SEQ ID NO: 10 - DNA, 19 bp, Artificial Sequence
  Description of Artificial Sequence Synthetic primer
atttcatgcttttgttctt

SEQ ID NO: 11 - DNA, 20 bp, Artificial Sequence
  Description of Artificial Sequence Synthetic primer
nataaaaagtcagcgaaaac

SEQ ID NO: 12 - DNA, 24 bp, Artificial Sequence
  Description of Artificial Sequence Synthetic primer
actcttggcagaagtttgagaaga

SEQ ID NO: 13 - DNA, 19 bp, Artificial Sequence
  Description of Artificial Sequence Synthetic primer
tggctttaaagaacttagg

SEQ ID NO: 14 - DNA, 21 bp, Artificial Sequence
  Description of Artificial Sequence Synthetic primer
naaagaggcttattaggtttc

SEQ ID NO: 15 - DNA, 25 bp, Artificial Sequence
  Description of Artificial Sequence Synthetic primer
cagtcgaagtcacctagttcttctg

SEQ ID NO: 16 - DNA, 17 bp, Artificial Sequence
  Description of Artificial Sequence Synthetic primer
cacccgttagaaaatgt

SEQ ID NO: 17 - DNA, 21 bp, Artificial Sequence
  Description of Artificial Sequence Synthetic primer
nttcaaaaacagatgtaagca

SEQ ID NO: 18 - DNA, 24 bp, Artificial Sequence
  Description of Artificial Sequence Synthetic primer
gttttgccatagcatgacacaatg

SEQ ID NO: 19 - DNA, 20 bp, Artificial Sequence
  Description of Artificial Sequence Synthetic primer
attgcatttggtttctttta

SEQ ID NO: 20 - DNA, 20 bp, Artificial Sequence
  Description of Artificial Sequence Synthetic primer
ngttttaccaatcgacatgt

SEQ ID NO: 21 - DNA, 33 bp, Artificial Sequence
  Description of Artificial Sequence Synthetic primer
ctattgttatgacactggtataccaacctgttc

SEQ ID NO: 22 - DNA, 20 bp, Artificial Sequence
  Description of Artificial Sequence Synthetic primer
atgacbacaatgatatgtgc

SEQ ID NO: 23 - DNA, 23 bp, Artificial Sequence
  Description of Artificial Sequence Synthetic primer
nctggtttactkacatctattga

SEQ ID NO: 24 - DNA, 23 bp, Artificial Sequence
  Description of Artificial Sequence Synthetic primer
agacatgcaccaccagaaacaaa

SEQ ID NO: 25 - DNA, 20 bp, Artificial Sequence
  Description of Artificial Sequence Synthetic primer
tgagtgattaagtttgatga

SEQ ID NO: 26 - DNA, 19 bp, Artificial Sequence
  Description of Artificial Sequence Synthetic primer
nattattaccyggaccaag

SEQ ID NO: 27 - DNA, 25 bp, Artificial Sequence
  Description of Artificial Sequence Synthetic primer
tgcatcaccaattgataatgaaggt

SEQ ID NO: 28 - DNA, 17 bp, Artificial Sequence
  Description of Artificial Sequence Synthetic primer
gggttgattgtggccca

SEQ ID NO: 29 - DNA, 32 bp, Artificial Sequence
  Description of Artificial Sequence Synthetic primer
ntgagacttgctttctattattataatgatac

SEQ ID NO: 30 - DNA, 30 bp, Artificial Sequence
  Description of Artificial Sequence Synthetic primer
tgccctgttgtgtttggaagagatatgact

SEQ ID NO: 31 - DNA, 20 bp, Artificial Sequence
  Description of Artificial Sequence Synthetic primer
atgcttatacctcraatcta

SEQ ID NO: 32 - DNA, 21 bp, Artificial Sequence
  Description of Artificial Sequence Synthetic primer
ntrggatttaagtcaggtacc

SEQ ID NO: 33 - DNA, 25 bp, Artificial Sequence
  Description of Artificial Sequence Synthetic primer
actcgaggttgycaggatataggaa

SEQ ID NO: 34 - DNA, 17 bp, Artificial Sequence
  Description of Artificial Sequence Synthetic primer
gggcgatttcaattttt

SEQ ID NO: 35 - DNA, 20 bp, Artificial Sequence
  Description of Artificial Sequence Synthetic primer
ngcagagggtcgattatata

SEQ ID NO: 36 - DNA, 33 bp, Artificial Sequence
  Description of Artificial Sequence Synthetic primer
cctctctgataataaaatatgttgttctcaatg

SEQ ID NO: 37 - DNA, 15 bp, Artificial Sequence
  Description of Artificial Sequence Synthetic primer
tgtgcaggtgctttc

SEQ ID NO: 38 - DNA, 17 bp, Artificial Sequence
  Description of Artificial Sequence Synthetic primer
ncccataaggcaagaag

SEQ ID NO: 39 - DNA, 34 bp, Artificial Sequence
  Description of Artificial Sequence Synthetic primer
caatgatcttttattttcgcaattatgtttgttt

SEQ ID NO: 40 - DNA, 16 bp, Artificial Sequence
  Description of Artificial Sequence Synthetic primer
attgcctgcaccatyt

SEQ ID NO: 41 - DNA, 16 bp, Artificial Sequence
  Description of Artificial Sequence Synthetic primer
ngttytggccagcact

SEQ ID NO: 42 - DNA, 23 bp, Artificial Sequence
  Description of Artificial Sequence Synthetic primer
acttgatccagccatttgctcca

SEQ ID NO: 43 - DNA, 20 bp, Artificial Sequence
  Description of Artificial Sequence Synthetic primer
aaagaagattcatcacagag

SEQ ID NO: 44 - DNA, 20 bp, Artificial Sequence
  Description of Artificial Sequence Synthetic primer
ntgctatttcaaatgcttca

SEQ ID NO: 45 - DNA, 25 bp, Artificial Sequence
  Description of Artificial Sequence Synthetic primer
caggaatgggaacaacagcaacaaa

SEQ ID NO: 46 - DNA, 18 bp, Artificial Sequence
  Description of Artificial Sequence Synthetic primer
magyactctgttgtcscc

SEQ ID NO: 47 - DNA, 16 bp, Artificial Sequence
  Description of Artificial Sequence Synthetic primer
ngggtctggtgcagtt

SEQ ID NO: 48 - DNA, 20 bp, Artificial Sequence
  Description of Artificial Sequence Synthetic primer
gtcaacgggcayraagcgca

SEQ ID NO: 49 - DNA, 16 bp, Artificial Sequence
  Description of Artificial Sequence Synthetic primer
ctgaagtacgtctcgg

SEQ ID NO: 50 - DNA, 16 bp, Artificial Sequence
  Description of Artificial Sequence Synthetic primer
ngctaccccttcgatg

SEQ ID NO: 51 - DNA, 16 bp, Artificial Sequence
  Description of Artificial Sequence Synthetic primer
ggcgtcctggcccgag

SEQ ID NO: 52 - DNA, 18 bp, Artificial Sequence
  Description of Artificial Sequence Synthetic primer
cattggcatagaggaagt

SEQ ID NO: 53 - DNA, 17 bp, Artificial Sequence
  Description of Artificial Sequence Synthetic primer
nagatgcaggttctgaa

SEQ ID NO: 54 - DNA, 28 bp, Artificial Sequence
  Description of Artificial Sequence Synthetic primer
aaggattgcctacatggatttcattagc

SEQ ID NO: 55 - DNA, 20 bp, Artificial Sequence
  Description of Artificial Sequence Synthetic primer
nattggacgatatcgttctc

SEQ ID NO: 56 - DNA, 20 bp, Artificial Sequence
  Description of Artificial Sequence Synthetic primer
aacggataatactaaaggcc

SEQ ID NO: 57 - DNA, 19 bp, Artificial Sequence
  Description of Artificial Sequence Synthetic probe
cccaatccacggacacagg

SEQ ID NO: 58 - DNA, 24 bp, Artificial Sequence
  Description of Artificial Sequence Synthetic primer
caattccttccccattcttttgac

SEQ ID NO: 59 - DNA, 25 bp, Artificial Sequence
  Description of Artificial Sequence Synthetic primer
nctatgaacacagcaaaaacaatga

SEQ ID NO: 60 - DNA, 15 bp, Artificial Sequence
  Description of Artificial Sequence Synthetic primer
ccgaggtcgaaacgt

SEQ ID NO: 61 - DNA, 15 bp, Artificial Sequence
  Description of Artificial Sequence Synthetic primer
cagaggtcgaaacgt

SEQ ID NO: 62 - DNA, 14 bp, Artificial Sequence
  Description of Artificial Sequence Synthetic primer
ntgggcacggtgag

SEQ ID NO: 63 - DNA, 23 bp, Artificial Sequence
  Description of Artificial Sequence Synthetic primer
ngcaacaaccaatccattaataa

SEQ ID NO: 64 - DNA, 18 bp, Artificial Sequence
  Description of Artificial Sequence Synthetic primer
ccagccatttgntccata

SEQ ID NO: 65 - DNA, 23 bp, Artificial Sequence
  Description of Artificial Sequence Synthetic primer
caaccattagaaaatgtctttat

SEQ ID NO: 66 - DNA, 19 bp, Artificial Sequence
  Description of Artificial Sequence Synthetic primer
nattattaccnggaccaag

SEQ ID NO: 67 - DNA, 33 bp, Artificial Sequence
  Description of Artificial Sequence Synthetic primer
nctgagacttgctttctattattataatgatac

SEQ ID NO: 68 - DNA, 20 bp, Artificial Sequence
  Description of Artificial Sequence Synthetic primer
atgcttatacctcaaatcta

SEQ ID NO: 69 - DNA, 20 bp, Artificial Sequence
  Description of Artificial Sequence Synthetic primer
atgcttatacctcgaatcta

SEQ ID NO: 70 - DNA, 21 bp, Artificial Sequence
  Description of Artificial Sequence Synthetic primer
ntaggatttaagtcaggtacc

SEQ ID NO: 71 - DNA, 21 bp, Artificial Sequence
  Description of Artificial Sequence Synthetic primer
ntgggatttaagtcaggtacc

SEQ ID NO: 72 - DNA, 18 bp, Artificial Sequence
  Description of Artificial Sequence Synthetic primer
ncggncacccaaagtagt

SEQ ID NO: 73 - DNA, 18 bp, Artificial Sequence
  Description of Artificial Sequence Synthetic primer
cagtactctgttgtcgcc

SEQ ID NO: 74 - DNA, 17 bp, Artificial Sequence
  Description of Artificial Sequence Synthetic primer
gaccnatggaattccca

SEQ ID NO: 75 - DNA, 21 bp, Artificial Sequence
  Description of Artificial Sequence Synthetic primer
ntgttctacnacactcattat

SEQ ID NO: 76 - DNA, 20 bp, Artificial Sequence
  Description of Artificial Sequence Synthetic primer
ngaaagaggcttattaggtt

SEQ ID NO: 77 - DNA, 19 bp, Artificial Sequence
  Description of Artificial Sequence Synthetic primer
catagcagcccgaggatct

SEQ ID NO: 78 - DNA, 21 bp, Artificial Sequence
  Description of Artificial Sequence Synthetic primer
ccggcncctgaatgnggctaa

SEQ ID NO: 79 - DNA, 18 bp, Artificial Sequence
  Description of Artificial Sequence Synthetic primer
gcccctgaatgcggctaa

SEQ ID NO: 80 - DNA, 21 bp, Artificial Sequence
  Description of Artificial Sequence Synthetic primer
aggatgccactaaacctcagc

SEQ ID NO: 81 - DNA, 25 bp, Artificial Sequence
  Description of Artificial Sequence Synthetic primer
cagtcgaagtcacctagttcttctg

SEQ ID NO: 82 - DNA, 24 bp, Artificial Sequence
  Description of Artificial Sequence Synthetic primer
actcttggcagaagtttgagaaga

SEQ ID NO: 83 - DNA, 23 bp, Artificial Sequence
  Description of Artificial Sequence Synthetic primer
gtnttccaaaatgcaatcagctg

SEQ ID NO: 84 - DNA, 20 bp, Artificial Sequence
  Description of Artificial Sequence Synthetic primer
gtcaacgggcacaaagcgca

SEQ ID NO: 85 - DNA, 16 bp, Artificial Sequence
  Description of Artificial Sequence Synthetic primer
ggcgtcctggcccgag

SEQ ID NO: 86 - DNA, 28 bp, Artificial Sequence
  Description of Artificial Sequence Synthetic primer
aaggattgcctacatggatttcattagc

SEQ ID NO: 87 - DNA, 28 bp, Artificial Sequence
  Description of Artificial Sequence Synthetic primer
aaggattgcctacatggatttcattagc

SEQ ID NO: 88 - DNA, 34 bp, Artificial Sequence; identical to SEQ ID NO: 39
  Description of Artificial Sequence Synthetic primer

SEQ ID NO: 89 - DNA, 29 bp, Artificial Sequence
  Description of Artificial Sequence Synthetic primer
aatggctaaagacaagaccaatcctgtca

SEQ ID NO: 90 - DNA, 24 bp, Artificial Sequence
  Description of Artificial Sequence Synthetic primer
gagctggccanaaccattctgttc

SEQ ID NO: 91 - DNA, 27 bp, Artificial Sequence
  Description of Artificial Sequence Synthetic primer
ggacgtcttctccttttcccattccat

SEQ ID NO: 92 - DNA, 24 bp, Artificial Sequence
  Description of Artificial Sequence Synthetic primer
gttttgccatagcatgacacaatg

SEQ ID NO: 93 - DNA, 33 bp, Artificial Sequence; identical to SEQ ID NO: 21
  Description of Artificial Sequence Synthetic primer

SEQ ID NO: 94 - DNA, 34 bp, Artificial Sequence
  Description of Artificial Sequence Synthetic primer
aattctattgttatgacactggtataccaacctg

SEQ ID NO: 95 - DNA, 25 bp, Artificial Sequence
  Description of Artificial Sequence Synthetic primer
actcgaggttgtcaggatataggaa

SEQ ID NO: 96 - DNA, 25 bp, Artificial Sequence
  Description of Artificial Sequence Synthetic primer
actcgaggttgccaggatataggaa

SEQ ID NO: 97 - DNA, 25 bp, Artificial Sequence
  Description of Artificial Sequence Synthetic primer
tgcatcaccaattgataatgaaggt

SEQ ID NO: 98 - DNA, 31 bp, Artificial Sequence
  Description of Artificial Sequence Synthetic primer
tgccctgttgtgtttggaagagatatgactc

SEQ ID NO: 99 - DNA, 26 bp, Artificial Sequence
  Description of Artificial Sequence Synthetic primer
atggctgaaaaagggaaaacaaatag

SEQ ID NO: 100 - DNA, 30 bp, Artificial Sequence
  Description of Artificial Sequence Synthetic primer
agcttaagatgtgattttacatattttagg

SEQ ID NO: 101 - DNA, 26 bp, Artificial Sequence
  Description of Artificial Sequence Synthetic primer
catggaagattacagcaatctatctc

SEQ ID NO: 102 - DNA, 27 bp, Artificial Sequence
  Description of Artificial Sequence Synthetic primer
ttaaagcattagttcccttaaaaatgg

SEQ ID NO: 103 - DNA, 25 bp, Artificial Sequence
  Description of Artificial Sequence Synthetic primer
atagacaaatccaaattcgagatgg

SEQ ID NO: 104 - DNA, 33 bp, Artificial Sequence
  Description of Artificial Sequence Synthetic primer
cttgtattatagatagattgatgcatattatgg

SEQ ID NO: 105 - DNA, 17 bp, Artificial Sequence
  Description of Artificial Sequence Synthetic primer
cccttgcatgtgcaacc

SEQ ID NO: 106 - DNA, 18 bp, Artificial Sequence
  Description of Artificial Sequence Synthetic primer
cgatgatgtgtcggatcg

SEQ ID NO: 107 - DNA, 18 bp, Artificial Sequence
  Description of Artificial Sequence Synthetic primer
cacttctcagctccaacc

SEQ ID NO: 108 - DNA, 18 bp, Artificial Sequence
  Description of Artificial Sequence Synthetic primer
ggcatgtggaggaacttg

SEQ ID NO: 109 - DNA, 19 bp, Artificial Sequence
  Description of Artificial Sequence Synthetic primer
caacacaacacgccagtag

SEQ ID NO: 110 - DNA, 28 bp, Artificial Sequence
  Description of Artificial Sequence Synthetic primer
tgtaagtgagatggtttatagataagag

SEQ ID NO: 111 - DNA, 23 bp, Artificial Sequence
  Description of Artificial Sequence Synthetic primer
gaacacacaatccaacagcaatc

SEQ ID NO: 112 - DNA, 21 bp, Artificial Sequence
  Description of Artificial Sequence Synthetic primer
ggattggtgatcagcagactg

SEQ ID NO: 113 - DNA, 21 bp, Artificial Sequence
  Description of Artificial Sequence Synthetic primer
atgagtcttctaaccgaggtc

SEQ ID NO: 114 - DNA, 21 bp, Artificial Sequence
  Description of Artificial Sequence Synthetic primer
ctgctgttcctctcgatattc

SEQ ID NO: 115 - DNA, 18 bp, Artificial Sequence
  Description of Artificial Sequence Synthetic primer
atgtcgctgtttggagac

SEQ ID NO: 116 - DNA, 23 bp, Artificial Sequence
  Description of Artificial Sequence Synthetic primer
cccatttttattatctcttcggc

SEQ ID NO: 117 - DNA, 23 bp, Artificial Sequence
  Description of Artificial Sequence Synthetic primer
tgaatccactgttaatgtctatc

SEQ ID NO: 118 - DNA, 24 bp, Artificial Sequence
  Description of Artificial Sequence Synthetic primer
tgctgtagtgtatagcactatcac

SEQ ID NO: 119 - DNA, 23 bp, Artificial Sequence
  Description of Artificial Sequence Synthetic primer
accagtatcttaagtctttcccc

SEQ ID NO: 120 - DNA, 22 bp, Artificial Sequence
  Description of Artificial Sequence Synthetic primer
tctctgcattccctaagttatc

SEQ ID NO: 121 - DNA, 21 bp, Artificial Sequence
  Description of Artificial Sequence Synthetic primer
tatggagtagcgcttaacttg

SEQ ID NO: 122 - DNA, 18 bp, Artificial Sequence
  Description of Artificial Sequence Synthetic primer
tcttgctcgctggagccg

SEQ ID NO: 123 - DNA, 20 bp, Artificial Sequence
  Description of Artificial Sequence Synthetic primer
aagcacactgaacagcatcg

SEQ ID NO: 124 - DNA, 19 bp, Artificial Sequence
  Description of Artificial Sequence Synthetic primer
agttcctgctcactggagc

SEQ ID NO: 125 - DNA, 19 bp, Artificial Sequence
  Description of Artificial Sequence Synthetic primer
cagaaggaggagtgaagag

SEQ ID NO: 126 - DNA, 18 bp, Artificial Sequence
  Description of Artificial Sequence Synthetic primer
ctggtgcactcggacgac

SEQ ID NO: 127 - DNA, 20 bp, Artificial Sequence
  Description of Artificial Sequence Synthetic primer
gtgacattgtcacccatttg

SEQ ID NO: 128 - DNA, 18 bp, Artificial Sequence
  Description of Artificial Sequence Synthetic primer
caaggcgccaaatctctc

SEQ ID NO: 129 - DNA, 20 bp, Artificial Sequence
  Description of Artificial Sequence Synthetic primer
actacaccagcaccagttta

SEQ ID NO: 130 - DNA, 19 bp, Artificial Sequence
  Description of Artificial Sequence Synthetic primer
agatgccgacataaggttc

SEQ ID NO: 131 - DNA, 19 bp, Artificial Sequence
  Description of Artificial Sequence Synthetic primer
cctgttccagctgaagtac

SEQ ID NO: 132 - DNA, 19 bp, Artificial Sequence
  Description of Artificial Sequence Synthetic primer
ccttatcatgcgctaaacg

SEQ ID NO: 133 - DNA, 17 bp, Artificial Sequence
  Description of Artificial Sequence Synthetic primer
cggtaccyttgtrcgcc

SEQ ID NO: 134 - DNA, 19 bp, Artificial Sequence
  Description of Artificial Sequence Synthetic primer
attgtcaccatwagcagyc

SEQ ID NO: 135 - DNA, 19 bp, Artificial Sequence
  Description of Artificial Sequence Synthetic primer
caagcacttctgtttcccc

SEQ ID NO: 136 - DNA, 22 bp, Artificial Sequence
  Description of Artificial Sequence Synthetic primer
tcaacagatggtgattgtagtg

SEQ ID NO: 137 - DNA, 21 bp, Artificial Sequence
  Description of Artificial Sequence Synthetic primer
cctatcagagtatccacaagc

SEQ ID NO: 138 - DNA, 21 bp, Artificial Sequence
  Description of Artificial Sequence Synthetic primer
tctacatacagaagtgtctgg

SEQ ID NO: 139 - DNA, 16 bp, Artificial Sequence
  Description of Artificial Sequence Synthetic primer
gtgtcaggcttggtgg

SEQ ID NO: 140 - DNA, 20 bp, Artificial Sequence
  Description of Artificial Sequence Synthetic primer
ctgcatgctgaagtatctgg

SEQ ID NO: 141 - DNA, 22 bp, Artificial Sequence
  Description of Artificial Sequence Synthetic primer
atctacatacagatgtgtctgg

SEQ ID NO: 142 - DNA, 21 bp, Artificial Sequence
  Description of Artificial Sequence Synthetic primer
gttgagttggtttgtcaatgg

SEQ ID NO: 143 - DNA, 22 bp, Artificial Sequence
  Description of Artificial Sequence Synthetic primer
atctattggttgaggtatcagg

SEQ ID NO: 144 - DNA, 20 bp, Artificial Sequence
  Description of Artificial Sequence Synthetic primer
ccatgttttgctctccaagg

SEQ ID NO: 145 - DNA, 18 bp, Artificial Sequence
  Description of Artificial Sequence Synthetic primer
agtgtcaggctgagtagg

SEQ ID NO: 146 - DNA, 20 bp, Artificial Sequence
  Description of Artificial Sequence Synthetic primer
ctatttgaggaggtgtctgg

SEQ ID NO: 147 - DNA, 22 bp, Artificial Sequence
  Description of Artificial Sequence Synthetic primer
actctctagagtgtaaaatcgg

SEQ ID NO: 148 - DNA, 25 bp, Artificial Sequence
  Description of Artificial Sequence Synthetic primer
ccacaactgactttctagtgtatag

SEQ ID NO: 149 - DNA, 18 bp, Artificial Sequence
  Description of Artificial Sequence Synthetic primer
taatttccagcaccagcc

SEQ ID NO: 150 - DNA, 22 bp, Artificial Sequence
  Description of Artificial Sequence Synthetic primer
tatctattgcagtagcatcctc

SEQ ID NO: 151 - DNA, 18 bp, Artificial Sequence
  Description of Artificial Sequence Synthetic primer
tagtttccaccaccaacc

SEQ ID NO: 152 - DNA, 25 bp, Artificial Sequence
  Description of Artificial Sequence Synthetic primer
ccatgttttgctttctaaggtataa

SEQ ID NO: 153 - DNA, 21 bp, Artificial Sequence
  Description of Artificial Sequence Synthetic primer
tctattggatgaagtgtcagg

SEQ ID NO: 154 - DNA, 19 bp, Artificial Sequence
  Description of Artificial Sequence Synthetic primer
gtagaggtgtctggttgtg

SEQ ID NO: 155 - DNA, 19 bp, Artificial Sequence
  Description of Artificial Sequence Synthetic primer
ccttctttgctttccagtg

SEQ ID NO: 156 - DNA, 20 bp, Artificial Sequence
  Description of Artificial Sequence Synthetic primer
tgaggaagtgtctggtctag

SEQ ID NO: 157 - DNA, 19 bp, Artificial Sequence
  Description of Artificial Sequence Synthetic primer
tgttggaagatgtgtcagg

SEQ ID NO: 158 - DNA, 21 bp, Artificial Sequence
  Description of Artificial Sequence Synthetic primer
agtgtgtagaatctgttggac

SEQ ID NO: 159 - DNA, 22 bp, Artificial Sequence
  Description of Artificial Sequence Synthetic primer
aaggtgtaaaatctgttggaag

SEQ ID NO: 160 - DNA, 20 bp, Artificial Sequence
  Description of Artificial Sequence Synthetic primer
agatgaagtgtctggttgtg

SEQ ID NO: 161 - DNA, 21 bp, Artificial Sequence
  Description of Artificial Sequence Synthetic primer
aatctacatgctgatgtgtcc

SEQ ID NO: 162 - DNA, 20 bp, Artificial Sequence
  Description of Artificial Sequence Synthetic primer
ggcaagtgtgtaaaatctgc

SEQ ID NO: 163 - DNA, 19 bp, Artificial Sequence
  Description of Artificial Sequence Synthetic primer
gaagaagtgtcagggtgtg

SEQ ID NO: 164 - DNA, 16 bp, Artificial Sequence
  Description of Artificial Sequence Synthetic primer
tgaggtgtctggctgg

SEQ ID NO: 165 - DNA, 23 bp, Artificial Sequence
  Description of Artificial Sequence Synthetic primer
ggtgtaaaacctattggaagatg

SEQ ID NO: 166 - DNA, 21 bp, Artificial Sequence
  Description of Artificial Sequence Synthetic primer
tctatttgatgaggtgtctgg

SEQ ID NO: 167 - DNA, 20 bp, Artificial Sequence
  Description of Artificial Sequence Synthetic primer
gaagtgtctggatgagaagg

SEQ ID NO: 168 - DNA, 23 bp, Artificial Sequence
  Description of Artificial Sequence Synthetic primer
ctattagaggaagtatctggtcg

SEQ ID NO: 169 - DNA, 22 bp, Artificial Sequence
  Description of Artificial Sequence Synthetic primer
atctattagaagaggtgtctgg

SEQ ID NO: 170 - DNA, 21 bp, Artificial Sequence
  Description of Artificial Sequence Synthetic primer
cctacatactgaagtgtctgg

SEQ ID NO: 171 - DNA, 20 bp, Artificial Sequence
  Description of Artificial Sequence Synthetic primer
agatgaagtgtctggttgtg

SEQ ID NO: 172 - DNA, 22 bp, Artificial Sequence
  Description of Artificial Sequence Synthetic primer
atctacatacagaagtgtctgg

SEQ ID NO: 173 - DNA, 19 bp, Artificial Sequence
  Description of Artificial Sequence Synthetic primer
atgtgtctggttgtgaagg

SEQ ID NO: 174 - DNA, 22 bp, Artificial Sequence
  Description of Artificial Sequence Synthetic primer
ccataccttactctctaaggtg

SEQ ID NO: 175 - DNA, 19 bp, Artificial Sequence
  Description of Artificial Sequence Synthetic primer
gatgatgtatcaggctggc

SEQ ID NO: 176 - DNA, 24 bp, Artificial Sequence
  Description of Artificial Sequence Synthetic primer
gaatctattggaagaagtatcagg

SEQ ID NO: 177 - DNA, 19 bp, Artificial Sequence
  Description of Artificial Sequence Synthetic primer
tacatgctgaagtgtctgg

SEQ ID NO: 178 - DNA, 20 bp, Artificial Sequence
  Description of Artificial Sequence Synthetic primer
ccatgacctactctccaaag

SEQ ID NO: 179 - DNA, 19 bp, Artificial Sequence
  Description of Artificial Sequence Synthetic primer
tatttgacgaggtgtctgg

SEQ ID NO: 180 - DNA, 21 bp, Artificial Sequence
  Description of Artificial Sequence Synthetic primer
cattccttactttccagtgtg

SEQ ID NO: 181 - DNA, 21 bp, Artificial Sequence
  Description of Artificial Sequence Synthetic primer
aggtatagaatcggttggatg

SEQ ID NO: 182 - DNA, 24 bp, Artificial Sequence
  Description of Artificial Sequence Synthetic primer
gaatctatttgatgaagtgtctgg

SEQ ID NO: 183 - DNA, 22 bp, Artificial Sequence
  Description of Artificial Sequence Synthetic primer
gtagaatctgcatacagatgtg

SEQ ID NO: 184 - DNA, 18 bp, Artificial Sequence
  Description of Artificial Sequence Synthetic primer
cggacgagaaggtttgtc

SEQ ID NO: 185 - DNA, 17 bp, Artificial Sequence
  Description of Artificial Sequence Synthetic primer
gtgtcaggacgtgatgg

SEQ ID NO: 186 - DNA, 21 bp, Artificial Sequence
  Description of Artificial Sequence Synthetic primer
cacaatttactgtccaaggtg

SEQ ID NO: 187 - DNA, 20 bp, Artificial Sequence
  Description of Artificial Sequence Synthetic primer
ccatgtcttgctgtccaatg

SEQ ID NO: 188 - DNA, 23 bp, Artificial Sequence
  Description of Artificial Sequence Synthetic primer
tcttgagcagataaatactctgg

SEQ ID NO: 189 - DNA, 20 bp, Artificial Sequence
  Description of Artificial Sequence Synthetic primer
gatgtgtcaggttgtgtagg

SEQ ID NO: 190 - DNA, 23 bp, Artificial Sequence
  Description of Artificial Sequence Synthetic primer
gatgaagtatctggtctagatgg

SEQ ID NO: 191 - DNA, 18 bp, Artificial Sequence
  Description of Artificial Sequence Synthetic primer
gttggaggaagtgtctgg

SEQ ID NO: 192 - DNA, 22 bp, Artificial Sequence
  Description of Artificial Sequence Synthetic primer
aggaagtatctggtctagatgg

SEQ ID NO: 193 - DNA, 22 bp, Artificial Sequence
  Description of Artificial Sequence Synthetic primer
aaggtgtagaatctattggagg

SEQ ID NO: 194 - DNA, 22 bp, Artificial Sequence
  Description of Artificial Sequence Synthetic primer
gatgatgtgtctggttgtgtag

SEQ ID NO: 195 - DNA, 21 bp, Artificial Sequence
  Description of Artificial Sequence Synthetic primer
tagatgatgtgtcaggttgtg

SEQ ID NO: 196 - DNA, 20 bp, Artificial Sequence
  Description of Artificial Sequence Synthetic primer
gatgtgtcaggcttagaagg

SEQ ID NO: 197 - DNA, 20 bp, Artificial Sequence
  Description of Artificial Sequence Synthetic primer
gaagtgtctggttgtgtagg

SEQ ID NO: 198 - DNA, 19 bp, Artificial Sequence
  Description of Artificial Sequence Synthetic primer
gtgtagaagcgattggagg

SEQ ID NO: 199 - DNA, 22 bp, Artificial Sequence
  Description of Artificial Sequence Synthetic primer
gtgtagaatctgtttgaggatg

SEQ ID NO: 200 - DNA, 22 bp, Artificial Sequence
  Description of Artificial Sequence Synthetic primer
tcggcatcagataagtattgtg

SEQ ID NO: 201 - DNA, 17 bp, Artificial Sequence
  Description of Artificial Sequence Synthetic primer
gcagtagcatctgcacc

SEQ ID NO: 202 - DNA, 23 bp, Artificial Sequence
  Description of Artificial Sequence Synthetic primer
agtgtagaatctgttagaagagg

SEQ ID NO: 203 - DNA, 21 bp, Artificial Sequence
  Description of Artificial Sequence Synthetic primer
tgaaacatctggatgagttgg

SEQ ID NO: 204 - DNA, 19 bp, Artificial Sequence
  Description of Artificial Sequence Synthetic primer
acctgcatactgacgtatc

SEQ ID NO: 205 - DNA, 20 bp, Artificial Sequence
  Description of Artificial Sequence Synthetic primer
ctacacactgatgtgtcagg

SEQ ID NO: 206 - DNA, 21 bp, Artificial Sequence
  Description of Artificial Sequence Synthetic primer
tctatttgaagaggtgtctgg

SEQ ID NO: 207 - DNA, 21 bp, Artificial Sequence
  Description of Artificial Sequence Synthetic primer
cccattttgctgtctaatgtg

SEQ ID NO: 208 - DNA, 22 bp, Artificial Sequence
  Description of Artificial Sequence Synthetic primer
ctattggaggaagtatcaggtc

SEQ ID NO: 209 - DNA, 18 bp, Artificial Sequence
  Description of Artificial Sequence Synthetic primer
agcagttgcatcttctgg

SEQ ID NO: 210 - DNA, 18 bp, Artificial Sequence
  Description of Artificial Sequence Synthetic primer
cggttgtgatggcttgtc

SEQ ID NO: 211 - DNA, 21 bp, Artificial Sequence
  Description of Artificial Sequence Synthetic primer
tctattagaggaggtgtctgg

SEQ ID NO: 212 - DNA, 17 bp, Artificial Sequence
  Description of Artificial Sequence Synthetic primer
acgtgtctggttgagtg

SEQ ID NO: 213 - DNA, 20 bp, Artificial Sequence
  Description of Artificial Sequence Synthetic primer
gctctccagagtatagaacc

SEQ ID NO: 214 - DNA, 20 bp, Artificial Sequence
  Description of Artificial Sequence Synthetic primer
ctacacacagatgtgtctgg

SEQ ID NO: 215 - DNA, 22 bp, Artificial Sequence
  Description of Artificial Sequence Synthetic primer
gtactttctagtgtgtacaacc

SEQ ID NO: 216 - DNA, 23 bp, Artificial Sequence
  Description of Artificial Sequence Synthetic primer
tatttgatgatgtatctggctgg

SEQ ID NO: 217 - DNA, 21 bp, Artificial Sequence
  Description of Artificial Sequence Synthetic primer
tctattggatgatgtgtcagg

SEQ ID NO: 218 - DNA, 20 bp, Artificial Sequence
  Description of Artificial Sequence Synthetic primer
ctgcacacagatgtatctgg

SEQ ID NO: 219 - DNA, 22 bp, Artificial Sequence
  Description of Artificial Sequence Synthetic primer
ccaagttttactagccaaagtg

SEQ ID NO: 220 - DNA, 23 bp, Artificial Sequence
  Description of Artificial Sequence Synthetic primer
ctatttgatgatgtgtcaggttg

SEQ ID NO: 221 - DNA, 28 bp, Artificial Sequence
  Description of Artificial Sequence Synthetic primer
tactatctaaggtgtaaaatctacacac

SEQ ID NO: 222 - DNA, 20 bp, Artificial Sequence
  Description of Artificial Sequence Synthetic primer
gaagtatctggttgtgttgg

SEQ ID NO: 223 - DNA, 18 bp, Artificial Sequence
  Description of Artificial Sequence Synthetic primer
gtccaagaacgactgtcc

SEQ ID NO: 224 - DNA, 22 bp, Artificial Sequence
  Description of Artificial Sequence Synthetic primer
ctattagatgaggtgtctggac

SEQ ID NO: 225 - DNA, 29 bp, Artificial Sequence
  Description of Artificial Sequence Synthetic primer
ttgctgtctaaagtataaaatctacatac

SEQ ID NO: 226 - DNA, 17 bp, Artificial Sequence
  Description of Artificial Sequence Synthetic primer
ctgcacactgaggtgtc

SEQ ID NO: 227 - DNA, 20 bp, Artificial Sequence
  Description of Artificial Sequence Synthetic primer
ctacatgcagatgtgtctgg

SEQ ID NO: 228 - DNA, 17 bp, Artificial Sequence
  Description of Artificial Sequence Synthetic primer
gttcgaggaagtgtcgg

SEQ ID NO: 229 - DNA, 22 bp, Artificial Sequence
  Description of Artificial Sequence Synthetic primer
atctattggttgaggtatcagg

SEQ ID NO: 230 - DNA, 19 bp, Artificial Sequence
  Description of Artificial Sequence Synthetic primer
ggaagatgtgtcaggtctg

SEQ ID NO: 231 - DNA, 21 bp, Artificial Sequence
  Description of Artificial Sequence Synthetic primer
tacaagcagatgtatcaggtc

SEQ ID NO: 232 - DNA, 20 bp, Artificial Sequence
  Description of Artificial Sequence Synthetic primer
tgaagatgtatctggttgcg

SEQ ID NO: 233 - DNA, 21 bp, Artificial Sequence
  Description of Artificial Sequence Synthetic primer
tctacaagctgaagtatctgg

SEQ ID NO: 234 - DNA, 21 bp, Artificial Sequence
  Description of Artificial Sequence Synthetic primer
tgttggaagatgtatcaggtg

SEQ ID NO: 235 - DNA, 20 bp, Artificial Sequence
  Description of Artificial Sequence Synthetic primer
tgaggaagtgtctggtctag

SEQ ID NO: 236 - DNA, 25 bp, Artificial Sequence
  Description of Artificial Sequence Synthetic primer
ncatggtgcagctgccgatctttgg

SEQ ID NO: 237 - DNA, 26 bp, Artificial Sequence
  Description of Artificial Sequence Synthetic primer
ntacatggtgcagctgccgatctttg

SEQ ID NO: 238 - DNA, 24 bp, Artificial Sequence
  Description of Artificial Sequence Synthetic primer
naatttacatggtgcagctgccaa

SEQ ID NO: 239 - DNA, 24 bp, Artificial Sequence
  Description of Artificial Sequence Synthetic primer
ngatttacatggttcaattgccga

SEQ ID NO: 240 - DNA, 29 bp, Artificial Sequence
  Description of Artificial Sequence Synthetic primer
acagttgaccctgcattctgacaatacca

SEQ ID NO: 241 - DNA, 29 bp, Artificial Sequence
  Description of Artificial Sequence Synthetic primer
acagtggatcctgcatttttacaatacca

SEQ ID NO: 242 - DNA, 25 bp, Artificial Sequence
  Description of Artificial Sequence Synthetic primer
ggggctgcttttactatccagcaag

SEQ ID NO: 243 - DNA, 25 bp, Artificial Sequence
  Description of Artificial Sequence Synthetic primer
ggagctgccttgattatccaacaag

SEQ ID NO: 244 - DNA, 25 bp, Artificial Sequence
  Description of Artificial Sequence Synthetic primer
ggagctgctttgattatccaacaag

SEQ ID NO: 245 - DNA, 22 bp, Artificial Sequence
  Description of Artificial Sequence Synthetic primer
ttgggtagtaaacagttgaccc

SEQ ID NO: 246 - DNA, 22 bp, Artificial Sequence
  Description of Artificial Sequence Synthetic primer
ttgggtagtaaacagtggatcc

SEQ ID NO: 247 - DNA, 26 bp, Artificial Sequence
  Description of Artificial Sequence Synthetic primer
tttgggtagtaaacagtggatcctgc

SEQ ID NO: 248 - DNA, 26 bp, Artificial Sequence
  Description of Artificial Sequence Synthetic primer
ttgggtagtaaacagtggatcctgca

SEQ ID NO: 249 - DNA, 27 bp, Artificial Sequence
  Description of Artificial Sequence Synthetic primer
ttgggtagtaaacagtagatcctgcat

SEQ ID NO: 250 - DNA, 25 bp, Artificial Sequence
  Description of Artificial Sequence Synthetic primer
ggggctgcttttactatccagcagg

(19) United States
(12) Patent Application Publication
(10) Pub. No.: US 2010/0086908 A1
(43) Pub. Date: Apr. 8, 2010

(54) METHODS FOR THE DETECTION OF RESPIRATORY VIRUSES

(76) Inventors: James R. Prudent, Madison, WI (US); David J. Marshall, Madison, WI (US); Wai-Ming Lee, Madison, WI (US); James Gern, Madison, WI (US); Michael J. Moser, Madison, WI (US); Edward L. Beaty, Madison, WI (US); Gerda Harms, Middleton, WI (US)

Correspondence Address: FOLEY & LARDNER LLP, 150 EAST GILMAN STREET, P.O. BOX 1497, MADISON, WI, 53701-1497, US

(21) Appl. No.: 12/122,250

(22) Filed: May 16, 2008

Related U.S. Application Data
(60) Provisional application No. 60/938,624, filed on May 17, 2007.

(51) Int. Cl. C12Q 1/70

(52) U.S. Cl. 435/5

(57) ABSTRACT

The present invention relates generally to the field of nucleic acid detection and more specifically to the detection of human respiratory viruses in a patient sample. In some aspects, the invention relates to the detection of multiple respiratory viral groups, including rhinovirus, respiratory syncytial virus, parainfluenza virus, influenza virus, metapneumovirus, adenovirus, coronavirus, and enterovirus.

### CROSS-REFERENCE TO RELATED APPLICATIONS

This application claims priority to U.S. Provisional Patent Application No. 60/938,624, filed May 17, 2007, which is incorporated by reference in its entirety.

### GOVERNMENT SUPPORT

This invention was made with United States Government support awarded by the following agencies: NIH AI025496. The United States Government has certain rights in this invention.

### FIELD OF THE INVENTION

The present invention relates generally to the field of nucleic acid detection and more specifically to the detection of human respiratory viruses in a patient sample. In some aspects, the invention relates to the detection of multiple respiratory viral groups, including rhinovirus, respiratory syncytial virus, parainfluenza virus, influenza virus, metapneumovirus, adenovirus, coronavirus, and enterovirus.

### BACKGROUND

Human respiratory viruses are a diverse group of pathogens which includes hundreds of different viral strains. Infection from such viral strains not only causes illness with symptoms including difficulty breathing, wheezing, coughing sneezing and fever, but also has been implicated as a risk factor for the development of childhood asthma. Respiratory viral infections are the most common illnesses in humans of all ages worldwide.

### SUMMARY

Disclosed herein are methods and compositions relating to the detection of respiratory viruses in patient samples. In some embodiments, assays are described wherein members of different groups of human respiratory viruses may be detected in a single assay, and such detection may be simultaneous or sequential.

In some aspects, a method of detecting a member of a human respiratory viral group in a sample may include the following steps: (a) reacting the sample and a reaction mixture to obtain an amplicon, the reaction mixture including a set of primer pairs specific for each viral group to be detected, wherein at least one primer of each pair includes at least one non-standard nucleobase, such as iso-C or iso-G; (b) hybridizing a target specific extension primer to the amplicon, wherein the target specific extension primer is different than any primer of the primer pairs, and wherein the target specific extension primer includes a tagging sequence that includes at least one non-standard nucleobase; (c) extending the hybridized target specific extension primer in the presence of a labeled nucleotide to obtain a labeled target oligonucleotide; (d) hybridizing the labeled target oligonucleotide to an immobilized oligonucleotide that hybridizes to the tagging sequence; and (e) detecting the labeled target oligonucleotide.

As noted above, members of more than one viral group may be detected simultaneously. In some embodiments, the human respiratory viruses to be detected in a given assay (e.g., a multiplex assay) may include, but are not limited to viruses from one or more of the following viral groups: rhinovirus (HRV), respiratory syncytial virus (RSV), parainfluenza virus (PIV), influenza virus (InfV), metapneumovirus (MPV), adenovirus (AdV), coronavirus (CoV), and enterovirus (EnV). In some embodiments, viruses from multiple different viral groups may be detected simultaneously as a set. For example, one set of viral groups may include the following: HRV, RSVA, RSVB, MPV, PIV1, PIV2, PIV3, PIV4a, PIV4b, InfVA and InfVB, CoVOC43, CoV229E, CoVNL63, AdVB, AdVC and AdVE, while another set may include the following viral groups: EnV, CoVOC43, CoV229E, CoVNL63, AdVB, AdVC and AdVE, and yet other set may include the following viral groups: HRV, RSVA, RSVB, MPV, PIV1, PIV2, PIV3, PIV4a, PIV4b, InfVA and InfVB. Still other embodiments may include a set including the following viral groups: EnV, CoVOC43, CoV229E, CoVNL63, AdVB, AdVC, AdVE, HRV, RSVA, RSVB, MPV, PIV1, PIV2, PIV3, PIV4a, PIV4b, InfVA and InfVB. In some embodiments, the viral groups of multiple sets may be detected simultaneously or sequentially.

In other aspects, a method of detecting a member of a human respiratory viral group in a sample may include the following steps: (a) reacting the sample and a first reaction mixture to obtain an amplicon, the first reaction mixture including a set of primer pairs specific for each viral group of a first set of human respiratory viral groups to be detected; (b) reacting the sample and a second reaction mixture to obtain an amplicon, the second reaction mixture including a set of primer pairs specific for each viral group of a second set of human respiratory viral groups to be detected, and wherein at least one primer of each pair comprises at least one non-standard nucleobase; (c) hybridizing a target specific primer to the amplicons of each set, wherein the target specific primer is different than any primer of the primer pairs and the target specific primer includes a tagging sequence that includes at least one non-standard nucleobase, such as iso-C or iso-G; (d) extending the hybridized target specific primer in the presence of a labeled nucleotide comprising a non-standard nucleobase that base-pairs with the nucleobase of the at least one primer to obtain a labeled target oligonucleotide; (e) hybridizing the labeled target oligonucleotide to an immobilized oligonucleotide that hybridizes to the tagging sequence; and (f) detecting the labeled target oligonucleotide.

In some embodiments, the first set of human respiratory viral groups may include HRV, RSVA, RSVB, MPV, PIV1, PIV2, PIV3, PIV4a, PIV4b, InfVA and InfVB, and the second set of human respiratory viral groups may include EnV, CoVOC43, CoV229E, CoVNL63, AdVB, AdVC and AdVE. In other embodiments, amplicons of the first and second viral groups may be generated simultaneously; in further embodiments, amplicons of the first and second viral groups may be generated sequentially. In still other embodiments, amplicons of the first and second viral groups may be generated in separate reaction vessels.

In further embodiments, the immobilized oligonucleotide may be coupled to a solid support; in some embodiments the solid support may include microspheres. In still other embodiments, the immobilized oligonucleotide may include one or more non-standard nucleobases, and the non-standard nucleobases of the tagging sequence may hybridize to the non-standard nucleobases of the immobilized oligonucleotide. In still other embodiments, the non-standard nucleobases of the immobilized oligonucleotide may be iso-C, iso-G or a combination of iso-C and iso-G.

In some embodiments, at least one primer of each primer pair may include at least one non-standard nucleobase. In some embodiments, the at least one non-standard nucleobase may be iso-C while in other embodiments, the at least one non-standard nucleobase may be iso-G. Thus, the at least one non-standard nucleobase of step (a) or step (b) may be iso-C, iso-G or both. Additionally or alternatively, a single primer may include more than one non-standard nucleobase (e.g., and may include, for example, both iso-G and iso-C), or one primer of the pair may include iso-C while the other may include iso-G. In still further embodiments, the primer pairs may include one or more of the sequences presented in Tables 3, 4, 7, 8, and 9.

In some embodiments, the reaction mixture used to generate the amplicons may include iso-C or iso-G nucleotide triphosphates; additionally or alternatively, the reaction mixture may include both iso-C and iso-G nucleotide triphosphates.

In some embodiments, the target specific extension primer may include a linker sequence; in other embodiments, the target specific extension primer may include a label. In still other embodiments, target specific extension primers may include one or more of the target specific extension (“TSE”) primer oligonucleotide sequences presented in Tables 4, 8 and 9. In some embodiments, the TSE primer may include at least one non-standard nucleobase. In some embodiments, the at least one non-standard nucleobase may be iso-C; in other embodiments, the at least one non-standard nucleobase may be iso-G. Additionally or alternatively, the TSE primer may include more than one non-standard nucleobase, and may include, for example, both iso-G and iso-C or multiple iso-Cs and/or multiple iso-Gs. In still other embodiments, the at least one non-standard nucleobase of the TSE primer may be present in the tag region, the analyte specific region or both.

In some embodiments, the TSE primer may be extended in the presence of non-standard nucleotide triphosphates, such as, for example iso-C or iso-G nucleotide triphosphates, or both iso-C and iso-G nucleotide triphosphates. In other embodiments, the non-standard nucleotide triphosphate may include a detectable label. In further embodiments, the label may be biotin and detection may include contacting fluorescent streptavidin-phycoerythrin (SAPE) with the biotin label.

In some embodiments, the sample may include a nasal wash sample.

### BRIEF DESCRIPTION OF THE DRAWINGS

The invention may be more completely understood in consideration of the following detailed description of various embodiments of the invention in connection with the accompanying drawings, in which:

FIG. 1 displays chemical structures for a number of non-standard bases, where A is the point of attachment to a polymeric backbone, X is N or C—Z, Y is N or C—H, and Z is H or a substituted or unsubstituted alkyl group;

FIGS. 2A and 2B schematically illustrate two examples of oligonucleotide hybridization to a solid support;

FIG. 3 illustrates steps in a second assay for an analyte-specific sequence, according to the invention;

FIG. 4 illustrates steps in a third assay for an analyte-specific sequence, according to the invention;

FIG. 5 illustrates steps in a fourth assay for an analyte-specific sequence, according to the invention;

FIG. 6 illustrates steps in a fifth assay for an analyte-specific sequence, according to the invention;

FIG. 7 illustrates steps in a sixth assay for an analyte-specific sequence, according to the invention;

FIG. 8 illustrates steps in a seventh assay for an analyte-specific sequence, according to the invention;

FIG. 9 illustrates steps in an eighth assay for an analyte-specific sequence, according to the invention;

FIG. 10 illustrates steps in a ninth assay for an analyte-specific sequence, according to the invention;

FIG. 11 illustrates steps in a tenth assay for an analyte-specific sequence, according to the invention;

FIGS. 12A-12D present data showing the detection of hMPV according to the invention;

FIG. 13 presents data showing the detection of hMPV according to the invention.

### DETAILED DESCRIPTION

The methods, kits and compositions described herein relate to the detection and identification of human respiratory viruses. Such viruses may include rhinovirus (HRV), respiratory syncytial virus (RSV), parainfluenza (PIV), influenza virus (infV), metapneumovirus (MPV), adenovirus (AdV), coronavirus (CoV) and enterovirus (EnV), and Bocavirus. In some aspects, multiple viral groups and/or members of multiple viral groups may be detected in a single assay.

The present invention is described herein using several definitions, as set forth below and throughout the specification.

As used herein, the term “subject” refers to an animal, preferably a mammal, more preferably a human. The term “subject” and “patient” may be used interchangeably.

As used herein, unless otherwise stated, the singular forms “a,” “an,” and “the” includes plural reference. Thus, for example, a reference to “an oligonucleotide” includes a plurality of oligonucleotide molecules, and a reference to “a nucleic acid” is a reference to one or more nucleic acids.

As used herein, when referring to a numerical value the term “about” means plus or minus 10% of the enumerated value, unless otherwise indicated.

As used herein, “nucleic acids” include polymeric molecules such as deoxyribonucleic acid (DNA), ribonucleic acid (RNA), peptide nucleic acid (PNA), or any sequence of what are commonly referred to as bases joined by a chemical backbone where the bases have the ability to form base pairs or hybridize with a complementary chemical structure. Suitable non-nucleotidic chemical backbones include, for example, polyamide and polymorpholino backbones. The term “nucleic acids” includes oligonucleotide, nucleotide, or polynucleotide sequences, and fragments or portions thereof. The nucleic acid can be provided in any suitable form, e.g., isolated from natural sources, recombinantly produced, or artificially synthesized, can be single- or double-stranded, and can represent the sense or antisense strand.

The term “oligonucleotide” refers generally to short chain (e.g., less than about 100 nucleotides in length, and typically 6 to 50 nucleotides in length) nucleic acid sequences as prepared using techniques presently available in the art such as, for example, solid support nucleic acid synthesis, DNA replication, reverse transcription, restriction digest, run-off transcription, or the like. The exact size of the oligonucleotide will typically depend upon a variety of factors, which in turn will depend upon the ultimate function or use of the oligonucleotide.

The nucleotides disclosed herein, which may include non-standard nucleotides, may be coupled to a label (e.g., a quencher or a fluorophore). Coupling may be performed using methods known in the art.

The oligonucleotides described herein may function as primers. In some embodiments, one or more oligonucleotides are labeled. For example, one or more oligonucleotides may be labeled with a reporter that emits a detectable signal (e.g., a fluorophore, a biotin, etc.). The oligonucleotides may include at least one non-standard nucleotide. For example, the oligonucleotides may include at least one nucleotide having a base that is not A, C, G, T, or U (e.g., iC or iG); in some embodiments, such non-standard base may be labeled. Where the oligonucleotide is used as a primer for PCR, the amplification mixture may include at least one non-standard nucleotide (e.g., iC or iG). Additionally or alternatively, the amplification mixture may include at least one non-standard nucleotide conjugated to a label (e.g., a fluorophore, a biotin, etc).

A “sequence” refers to an ordered arrangement of nucleotides.

The term “sample” includes a specimen or culture (e.g., microbiological cultures), as well as biological samples, samples derived from biological fluids, and samples from non-biological sources.

The term “analyte” refers to a nucleic acid suspected to be in a sample. The analyte is the object of the assay (e.g., the assay determines the presence, absence, concentration, or amount of the analyte in the sample). The analyte can be directly or indirectly assayed. In at least some embodiments involving indirect assay, the analyte, if present in the sample, is used as a template to form target oligonucleotides using, for example, PCR techniques. The target oligonucleotides are then assayed to indicate the presence, absence, concentration, or amount of the analyte in the sample.

The term “target oligonucleotide” refers to oligonucleotides that are actually assayed during an assay procedure. The target oligonucleotide can be, for example, an analyte or it can be an oligonucleotide containing an analyte-specific sequence that is the same as or complementary to a sequence of the analyte. For example, the target oligonucleotide can be a product of PCR amplification of an analyte or a portion of an analyte. In some embodiments, at least a portion of the target oligonucleotide may correspond to: a) the analyte, b) a portion of the analyte, c) a complement of the analyte, or d) a complement of a portion of the analyte. Detection of the target oligonucleotide by the assay indicates presence of the analyte in the original sample.

The term “capture oligonucleotide” refers to an oligonucleotide having a molecular recognition sequence and which may be coupled to a solid surface to hybridize with a target oligonucleotide having a tagging sequence or an analyte specific sequence complementary to the molecular recognition sequence, thereby capturing the target oligonucleotide on the solid surface.

A “molecular recognition sequence” as used herein is an oligonucleotide sequence complementary to the tagging sequence or to the analyte-specific sequence of a target oligonucleotide.

As used herein, the terms “complementary” or “complementarity,” when used in reference to nucleic acids (i.e., a sequence of nucleotides such as an oligonucleotide or a target nucleic acid), refer to sequences that are related by base-pairing rules. For natural bases, the base pairing rules are those developed by Watson and Crick. For non-standard bases, as described herein, the base-pairing rules refer to the formation of hydrogen bonds in a manner similar to the Watson-Crick base pairing rules or the formation of specific base pairs by hydrophobic, entropic, or van der Waals forces. As an example, for the sequence “T-G-A,” the complementary sequence is “A-C-T.” Complementarity can be “partial,” in which only some of the bases of the nucleic acids are matched according to the base pairing rules. Alternatively, there can be “complete” or “total” complementarity between the nucleic acids. The degree of complementarily between the nucleic acid strands affects the efficiency and strength of hybridization between the nucleic acid strands.

The term “hybridization” is used in reference to the pairing of complementary nucleic acids. Hybridization and the strength of hybridization (i.e., the strength of the association between the nucleic acids) is influenced by such factors as the degree of complementarity between the nucleic acids, stringency of the hybridization conditions involved, the melting temperature (Tm) of the formed hybrid, and the G:C ratio within the nucleic acids. Such factors are well known by those skilled in the art.

“Specific hybridization” is an indication that two nucleic acid sequences share a high degree of complementarity. Specific hybridization complexes form under permissive annealing conditions and remain hybridized after any subsequent washing steps. Permissive conditions for annealing of nucleic acid sequences are routinely determinable by one of ordinary skill in the art and may occur, for example, at 65° C. in the presence of about 6×SSC. Stringency of hybridization may be expressed, in part, with reference to the temperature under which the wash steps are carried out. Such temperatures are typically selected to be about 5° C. to 20° C. lower than the thermal melting point (Tm) for the specific sequence at a defined ionic strength and pH. The Tm is the temperature (under defined ionic strength and pH) at which 50% of the target sequence hybridizes to a perfectly matched probe. Equations for calculating Tm and conditions for nucleic acid hybridization are known in the art.

As used herein, “low stringency conditions” are selected to be about 10° C. to 15° C. below the thermal melting point (Tm) for the specific sequence at the ionic strength and pH of the hybridizing solution. Tm is the temperature (for the ionic strength, pH, and nucleic acid concentration) at which about 50% of the tagging sequences hybridize to complementary molecular recognition sequences at equilibrium.

As used herein, “moderate stringency conditions” are selected to be about 5° C. to 10° C. below the thermal melting point (Tm) for the specific sequence at the ionic strength and pH of the hybridizing solution.

As used herein, “high stringency conditions” are selected to be no more than about 5° C. below the thermal melting point (Tm) for the specific sequence at the ionic strength and pH of the hybridizing solution.

The present disclosure describes assays which may be performed to determine whether a sample includes an analyte having a particular nucleic acid sequence (or its complement). This nucleic acid sequence will be referred to as the “analyte-specific sequence.” In at least some instances, the original sample is not directly assayed. Instead, the analyte, if present, is cloned or amplified (e.g., by PCR techniques) to provide an assay sample with a detectable amount of a target oligonucleotide that contains the analyte-specific sequence.

As used herein, “amplification” or “amplifying” refers to the production of additional copies (“amplicons”) of a nucleic acid sequence. Amplification is generally carried out using polymerase chain reaction (PCR) technologies known in the art. The term “amplification reaction system” refers to any in vitro means for multiplying the copies of a target sequence of nucleic acid. The term “amplification reaction mixture” refers to an aqueous solution comprising the various reagents used to amplify a target nucleic acid. These may include enzymes (e.g., a thermostable polymerase), aqueous buffers, salts, amplification primers, target nucleic acid, and nucleoside triphosphates, and optionally at least one labeled probe and/or optionally at least one agent for determining the melting temperature of an amplified target nucleic acid (e.g., a fluorescent intercalating agent that exhibits a change in fluorescence in the presence of double-stranded nucleic acid).

Amplification of nucleic acids may include amplification of nucleic acids or subregions of these nucleic acids. For example, amplification may include amplifying portions of nucleic acids between 100 and 300 bases long by selecting the proper primer sequences and using the PCR.

One variety of PCR that may be used for some of the assays described below is “fast-shot PCR” in which primer extension times are reduced or eliminated. As used herein, the term “fast-shot polymerase chain reaction” or “fast-shot PCR” refers to PCR where the extension step, as well as the steps for the annealing and melting steps, are very short or eliminated. Typically, for this method, the 3′ ends of the two primers are separated by no more than 10 bases on the template nucleic acid.

In some embodiments, the PCR solution is rapidly cycled between about 90° C. to 100° C. and about 55° C. to 65° C. with a maximum of about a one second hold at each temperature, thereby leaving the polymerase very little time to extend mismatched primers. In one embodiment, the reaction is cycled between about 95° C. and about 58° C. with about a one second hold at each temperature.

This rapid cycling is facilitated by generating a short PCR product by, in general, leaving a gap of about zero (0) to ten (10) bases on the template nucleic acid between the 3′ bases of the first and second primers. Preferably, the primers are designed to have a Tm of approximately 55° C. to 60° C. For some embodiments, a total of about 37 cycles is typically adequate to detect as little as 30 target oligonucleotides.

Amplification mixtures may include natural nucleotides (including A, C, G, T, and U) and non-standard nucleotides (e.g., including iC and iG). The natural or non-standard bases used herein can be derivatized by substitution at non-hydrogen bonding sites to form modified natural or non-standard bases. For example, a base can be derivatized for attachment to a support by coupling a reactive functional group (for example, thiol, hydrazine, alcohol, amine, and the like) to a non-hydrogen bonding atom of the base. Other possible substituents include, for example, biotin, digoxigenin, fluorescent groups, alkyl groups (e.g., methyl or ethyl), and the like.

The methods disclosed herein may include transcription of RNA to DNA (i.e., reverse transcription). For example, reverse transcription may be performed prior to amplification.

Referring to oligonucleotides and bases, DNA and RNA are oligonucleotides that include deoxyriboses or riboses, respectively, coupled by phosphodiester bonds. Each deoxyribose or ribose includes a base coupled to a sugar. The bases incorporated in naturally-occurring DNA and RNA are adenosine (A), guanosine (G), thymidine (T), cytidine (C), and uridine (U). These five bases are “natural bases.” According to the rules of base pairing elaborated by Watson and Crick, the natural bases can hybridize to form purine-pyrimidine base pairs, where G pairs with C and A pairs with T or U. These pairing rules facilitate specific hybridization of an oligonucleotide with a complementary oligonucleotide.

The formation of these base pairs by the natural bases is facilitated by the generation of two or three hydrogen bonds between the two bases of each base pair. Each of the bases includes two or three hydrogen bond donor(s) and hydrogen bond acceptor(s). The hydrogen bonds of the base pair are each formed by the interaction of at least one hydrogen bond donor on one base with a hydrogen bond acceptor on the other base. Hydrogen bond donors include, for example, heteroatoms (e.g., oxygen or nitrogen) that have at least one attached hydrogen. Hydrogen bond acceptors include, for example, heteroatoms (e.g., oxygen or nitrogen) that have a lone pair of electrons.

The natural bases, A, G, C, T, and U, can be derivatized by substitution at non-hydrogen bonding sites to form modified natural bases. For example, a natural base can be derivatized for attachment to a support by coupling a reactive functional group (e.g., thiol, hydrazine, alcohol, or amine) to a non-hydrogen bonding atom of the base. Other possible substituents include biotin, digoxigenin, fluorescent groups, and alkyl groups (e.g., methyl or ethyl).

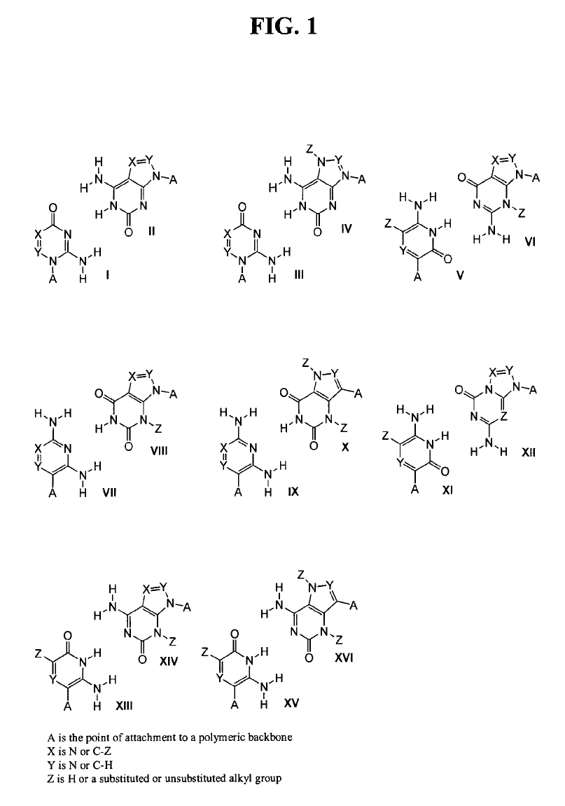

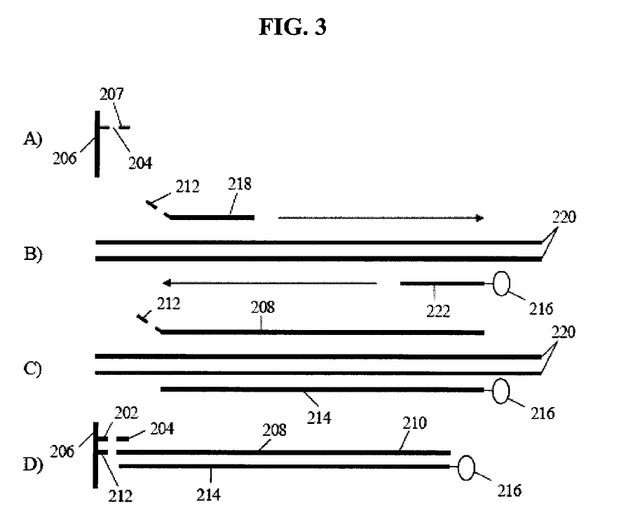

Non-standard bases, or non-standard bases, which form hydrogen-bonding base pairs, can also be constructed as described, for example, in U.S. Pat. Nos. 5,432,272, 5,965,364, 6,001,983, and 6,037,120 and U.S. patent application Ser. No. 08/775,401, all of which are incorporated herein by reference. By “non-standard base” it is meant a base other than A, G, C, T, or U that is susceptible of incorporation into an oligonucleotide and which is capable of base-pairing by hydrogen bonding, or by hydrophobic, entropic, or van der Waals interactions to form base pairs with a complementary base. FIG. 1 illustrates several examples of suitable bases and their corresponding base pairs. Specific examples of these bases include the following bases in base pair combinations (iso C/iso G, K/X, H/J, and M/N):

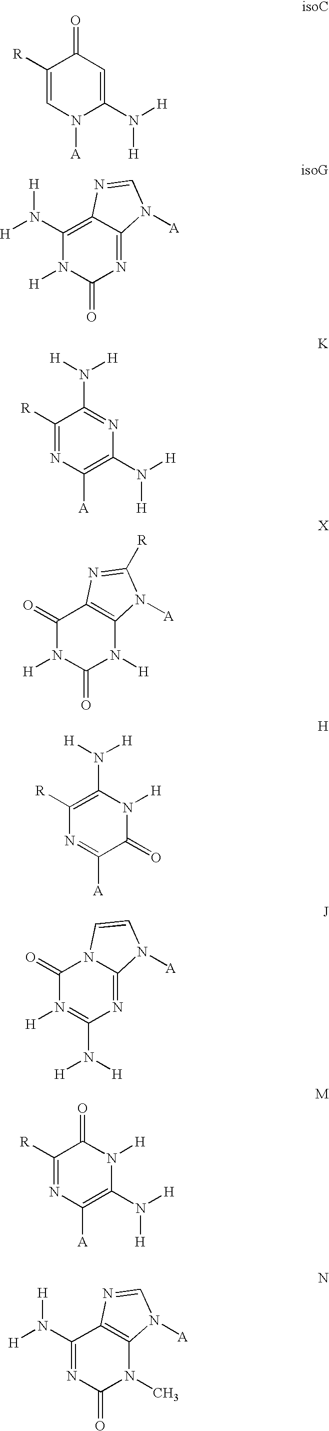

where A is the point of attachment to the sugar or other portion of the polymeric backbone and R is H or a substituted or unsubstituted alkyl group. It will be recognized that other non-standard bases utilizing hydrogen bonding can be prepared, as well as modifications of the above-identified non-standard bases by incorporation of functional groups at the non-hydrogen bonding atoms of the bases. To designate these non-standard bases in FIGS. 3 to 9, the following symbols will be used: X indicates iso-C and Y indicates iso-G.

The hydrogen bonding of these non-standard base pairs is similar to those of the natural bases where two or three hydrogen bonds are formed between hydrogen bond acceptors and hydrogen bond donors of the pairing non-standard bases. One of the differences between the natural bases and these non-standard bases is the number and position of hydrogen bond acceptors and hydrogen bond donors. For example, cytosine can be considered a donor/acceptor/acceptor base with guanine being the complementary acceptor/donor/donor base. Iso-C is an acceptor/acceptor/donor base and iso-G is the complementary donor/donor/acceptor base, as illustrated in U.S. Pat. No. 6,037,120, incorporated herein by reference.

Other non-standard bases for use in oligonucleotides include, for example, naphthalene, phenanthrene, and pyrene derivatives as discussed, for example, in Ren et al., J. Am. Chem. Soc. 118, 1671 (1996) and McMinn et al., J. Am. Chem. Soc. 121, 11585 (1999), both of which are incorporated herein by reference. These bases do not utilize hydrogen bonding for stabilization, but instead rely on hydrophobic, entropic, or van der Waals interactions to form base pairs.

As used herein, “labels” or “reporters” or “reporter molecules” are chemical or biochemical moieties useful for labeling a nucleic acid, amino acid, or antibody. “Labels” and “reporter molecules” include fluorescent agents, chemiluminescent agents, chromogenic agents, quenching agents, radionuclides, enzymes, substrates, cofactors, inhibitors, scintillation agents, magnetic particles, and other moieties known in the art. “Labels” or “reporter molecules” are capable of generating a measurable signal and may be covalently or noncovalently joined to an oligonucleotide.

As used herein, a “fluorescent dye” or a “fluorophore” is a chemical group that can be excited by light to emit fluorescence. Some suitable fluorophores may be excited by light to emit phosphorescence. Dyes may include acceptor dyes that are capable of quenching a fluorescent signal from a fluorescent donor dye. Fluorescent dyes or fluorophores may include derivatives that have been modified to facilitate conjugation to another reactive molecule. As such, fluorescent dyes or fluorophores may include amine-reactive derivatives such as isothiocyanate derivatives and/or succinimidyl ester derivatives of the fluorophore.

The oligonucleotides and nucleotides of the disclosed methods may be labeled with a quencher. Quenching may include dynamic quenching (e.g., by FRET), static quenching, or both. Suitable quenchers may include Dabcyl. Suitable quenchers may also include dark quenchers, which may include black hole quenchers sold under the tradename “BHQ” (e.g., BHQ-0, BHQ-1, BHQ-2, and BHQ-3, Biosearch Technologies, Novato, Calif.). Dark quenchers also may include quenchers sold under the tradename “QXL™” (Anaspec, San Jose, Calif.). Dark quenchers also may include DNP-type non-fluorophores that include a 2,4-dinitrophenyl group.

In some embodiments, the oligonucleotide of the present methods may be labeled with a donor fluorophore and an acceptor fluorophore (or quencher dye) that are present in the oligonucleotides at positions that are suitable to permit FRET (or quenching).

The term “viral group” or “virus group” with reference to a particular viral family or genus is meant to encompass all species, strains, types, serotypes, etc. within the family or genus that are considered respiratory pathogens. With respect to a species, “viral group” is meant to encompass all strains, types, serotypes, etc. within that species that are considered respiratory pathogens. With respect to a strain or serotype, “viral group” is meant to encompass all variants, mutants, subtypes, etc. within that strain that are considered respiratory pathogens. Non-limiting examples are described below.

For example, the viral group “rhinovirus” (“HRV”) is meant to include all viruses within the genus rhinovirus that are considered respiratory pathogens, including all 101 classically defined HRV serotypes, as well as newly discovered Group C HRV strains (currently numbering at least 46). Many of the Group C strains were first discovered using the assay described in this patent application. Exemplary rhinoviruses include but are not limited to serotypes 1B, 2, 9, 14, 16, 85 and 89, HRV serotype 1A, 1B, 2-10, 12-100, Hanks, HRV11, HRV90.

The viral group “respiratory syncytial virus” (“RSV”) is meant to include all types and serotypes (serotypes A and B) of the species respiratory syncytial virus that are considered respiratory pathogens. The viral group “RSVA” is meant to include all RSVA variants, mutants and subtypes.

The viral group “parainfluenza virus” (PIV) is meant to include any of the serotypes belonging to the family Paramyxoviridae that are considered respiratory pathogens. Exemplary parainfluenza viruses include but are not limited to PIV1, PIV2 and PIV3, PIV4a and PIV4b. Similarly, the viral group “PIV4a” is meant to include any mutants or variants of PIV4a.

The viral group “influenza virus” (InfV) is meant to include all viruses within any of the genera of influenza A, influenza B, and influenza C viruses that are considered respiratory pathogens. Exemplary influenza viruses include but are not limited to influenza A such as Sydney/05/97-like, H3N2, H5N1, influenza B such as Beijing/184/93-like.

The viral group “metapneumovirus” (MPV) is meant to include all viruses within the genus metapneumovirus that are considered respiratory pathogens. An exemplary metapneumovirus includes but is not limited to MPV isolate CAN97-83.

The viral group “adenovirus” (AdV) is meant to include all viruses within the family Adenoviridae that are considered respiratory pathogens. Exemplary adenoviruses include AdV 1, 2, 3, 4, 5, 6, 7, 11, 14, 16, 21, 34, 35.

The viral group “coronavirus” (CoV) is meant to include all viruses within the genus coronavirus that are considered respiratory pathogens. Exemplary CoV viruses include CoV 229E, CoV NL63, CoV OC43, and CoV HKU1.

The viral group “enterovirus” (EnV) is meant to include all viruses within the genus enterovirus that are considered respiratory pathogens. Exemplary enteroviruses include EV68-73, coxsackievirus A22, coxsackievirus B1.

In some embodiments, some or all members of different viral groups may be detected, if present in a sample, in a single assay. By way of example but not by way of limitation, some or all members of viral groups HRV, RSVA, RSVB, MPV, PIV1, PIV2, PIV3, PIV4a, PIV4b, InfVA and InfVB may be detected in a single assay. As another non-limiting example, some or all members of viral groups Env, CoVOC43, CoV229E, CoVNL63, AdVB, adVC and AdVE may be detected, if present in a sample, in a single assay. As a third non-limiting example, some or all members of viral groups HRV, RSVA, RSVB, MPV, PIV1, PIV2, PIV3, PIV4a, PIV4b, InfVA and InfVB, CoVOC43, CoV229E, CoVNL63, AdVB, AdVC and AdVE, may be detected if present in a sample, in a single assay. In yet another non-limiting example, some or all members of any two or more of the following viral groups may be detected in a single assay (e.g., a single multiplex reaction): HRV, RSVA, RSVB, MPV, PIV1, PIV2, PIV3, PIV4a, PIV4b, InfVA, InfVB, Env, CoVOC43, CoV229E, CoVNL63, AdVB, adVC and AdVE and bocavirus. Some sets of viral groups and/or their members may be detected simultaneously or sequentially.

As used herein, the term “allele” means an alternative form of a gene or a sequence. With respect to viral groups, an “allele” can be, for example, a single base mutation that distinguishes one virus in a viral group from another; such mutation need not be in a gene per se, but at some location in the viral genome.

As used herein, the term “linker sequence” or “linker region” refers to a nucleic acid sequence which does not hybridize to an analyte specific sequence (e.g., a respiratory viral sequence) or a tagging sequence under stringent conditions, but which may join or “link” such regions together, for example, on a TSE primer. A linker may be additional nucleotides or any other chemical linking moiety. In some embodiments a linker does not act as a template for oligonucleotide synthesis. In such embodiments, examples of suitable linkers include but are not limited to n-propyl, triethylene glycol, hexaethylene glycol, 1′, 2′ dideoxyribose, 2′-O-methylribonucleotides, deoxyisocytidine, or any linkage that would halt a polymerase. Thus in some embodiments, a TSE primer may include an analyte specific region, a linker sequence, and a tagging sequence.

As used herein, the term “target specific extension primer” (“TSE primer”) means an oligonucleotide which includes at minimum: 1) an analyte-specific 3′ region (e.g., a viral specific region) which will hybridize to the analyte sequence under stringent conditions; 2) a 5′ tag region, which does not hybridize to the analyte sequence under stringent conditions. Optionally, a TSE primer may also include a linker region. Thus in some embodiments, a TSE primer may be extended by PCR when hybridized to the analyte specific region of viral specific sequence. The term “target specific extension” (“TSE”) refers to an extension reaction using the TSE primer.

As used herein, the term “Multi-Code primer pair,” or “Multi-Code PCR primer pair,” is meant to include two primers which can be used to generate an amplicon, wherein at least one of the primers of the primer pair includes at least one non-standard nucleotide such as iso-C or iso-G. The term “Multi-Code primer” or “Multi-Code PCR primer” refers to a primer which is a member of “Multi-Code primer pair.” Thus, a single MultiCode primer may or may not include a non-standard nucleotide. A “Multi-Code primer set” refers to a “Multi-Code primer pair” and at least one additional oligonucleotide, such as a TSE oligonucleotide.

As used herein, the term “MultiCode PLx assay” means an assay which uses MultiCode primers or primer pairs or a primer set, and includes, at minimum, the following steps: 1) cDNA generation, 2) PCR amplification using MultiCode primers, 3) target specific extension and 4) capture of extension products.

As used herein, the term “Respiratory MultiCode Assay (“RMA”) mean an assay designed to detect respiratory viruses using MultiCode primers and MultiCode assay as described above.

Assays are performed to determine whether a sample includes an analyte having a particular nucleic acid sequence (or its complement). This nucleic acid sequence will be referred to as the “analyte-specific sequence”. In at least some instances, the original sample is not directly assayed. Instead, the analyte, if present, is cloned or amplified (e.g., by PCR techniques) to provide an assay sample with a detectable amount of a target oligonucleotide that contains the analyte-specific sequence. Other techniques for amplification include, for example, nucleic acid sequence based amplification, strand displacement amplification, incorporated herein by reference), ligase chain reaction, transcription mediated amplification, and rolling circle amplification. At least a portion of the target oligonucleotide typically corresponds to either a) the analyte, b) a portion of the analyte, c) a complement of the analyte, or d) a complement of a portion of the analyte. Detection of the target oligonucleotide by the assay indicates presence of the analyte in the original sample.

In general, an assay system for detecting one or more analyte-specific sequences includes a solid support (e.g., a chip, wafer, or a collection of solid particles). Capture oligonucleotides are disposed on the solid support in a manner which permits identification of the capture oligonucleotide (e.g., by position on a chip or wafer or by unique characteristic of particles to which particular capture oligonucleotides are attached). The capture oligonucleotides include a molecular recognition sequence. Different capture oligonucleotides with different molecular recognition sequences are used to detect different analyte-specific sequences. Using these different capture oligonucleotides, a single assay system can be designed to analyze a sample for multiple analyte-specific sequences.

Target oligonucleotides containing the analyte-specific sequences are brought into contact with the capture oligonucleotides. In addition to the analyte-specific sequence, the target oligonucleotides also each include a tagging sequence. A particular tagging sequence is associated with each analyte-specific sequence. The tagging sequence is generally complementary to one of the molecular recognition sequences. Thus, under hybridization conditions, the target oligonucleotides hybridize with the appropriate capture oligonucleotides. Alternatively, in certain methods, the analyte-specific sequence may be complementary to one of the molecular recognition sequences.

The target oligonucleotide or its complement typically includes a reporter or a coupling agent for attachment of a reporter. Observation of the solid support to determine the presence or absence of the reporter associated with a particular capture oligonucleotide indicates whether a particular analyte-specific sequence is present in the sample. Suitable reporters include, without limitation, biotin, fluorescents, chemilluminescents, digoxigenin, spin labels, radio labels, DNA cleavage moieties, chromaphors or fluoraphors. Examples of suitable coupling moieties include, but are not limited to, amines, thiols, hydrosines, alcohols or alkyl groups.

Exemplary assay system embodiments are described below. It is understood that the following descriptions are meant to aid the reader in understanding the invention and are not meant to be limiting.

In one embodiment, a sample containing an analyte (e.g., viral sequence or sequences of interest) is subject to RT-PCR or PCR to generate cDNA. The cDNA is further amplified by PCR using at least two primers specific for the analyte specific sequence to generate target oligonucleotide. At least one of these primers includes at least one non-standard base, such as iso-G or iso-C. Additionally, at least one of these primers includes a 3′ region specific for the analyte specific sequence (“viral-specific” region) and a 5′ tagging sequence which does not hybridize to the analyte specific sequence. In some embodiments, each tagging sequence is associated with a particular analyte (e.g., virus or virus group). For example, tagging sequence 1 is associated with virus group 1 and tagging sequence 2 is associated with virus group 2, etc., because tagging sequence 1 it is part of an oligonucleotide which will hybridize to virus group 1; tagging sequence 2 is part of an oligonucleotide that will hybridize to virus group 2, etc. Extension of such a primer is termed “target-specific extension” or “TSE,” and such primers may be termed “target specific extension primers” or “TSE” primers. Optionally, the TSE primer or the other primer may include a label, such as biotin. In some embodiments, a non-standard base may be incorporated into the tagging sequence of the TSE primer, the analyte specific region of the TSE primer or both. Additionally or alternatively, the non-TSE primer may include one or more non-standard bases. The amplification/extension reactions may be performed in the presence of non-standard nucleotides, thereby allowing the incorporation of non-standard nucleotides into the target oligonucleotides. In other embodiments, the amplification/extension reaction may be performed in the presence of a non-standard nucleotide conjugated to a detectable label or reporter, such as biotin. Accordingly, amplification with these primers produces target oligonucleotides which include a tagging sequence used in the “capture” step, a non-natural nucleotide, and/or a non-natural nucleotide conjugated to a detectable label or reporter such a biotin.

Capture oligonucleotides, designed to include a sequence complementary to the target oligonucleotide (the molecular recognition sequence) are conjugated to a solid support, such as a microsphere. The conjugated capture oligonucleotides are then contacted with the target oligonucleotides, and hybridization between the capture oligonucleotide molecular recognition sequence and the target oligonucleotide tagging sequence allows for the “capture” of the target oligonucleotide. The detectable label or reporter may then be detected by methods known in the art for identification and quantification of the targets.

In another embodiment, a patient sample containing an analyte (e.g., a viral sequence or sequences) of interest is subject to RT-PCR or PCR using random primers to generate cDNA. The cDNA is further amplified by PCR using primers specific for the analyte specific sequence. In some embodiments, at least one of the PCR primers includes one or more non-standard bases (e.g., iso-C, iso-G), and, optionally, the amplification reaction is performed in the presence of non-standard nucleotides, thereby allowing the incorporation of non-standard nucleotides into the amplicons. These amplicons are then interrogated by introducing at least one TSE primer which includes a 3′ region specific for the analyte specific sequence (e.g., “viral-specific” region) and a 5′ tagging sequence which does not hybridize to the analyte specific sequence. The TSE primer may additionally include a reporter group, such as biotin. Extension with the TSE primer produces tagged target oligonucleotides as described above with the tagging sequence being used in the capture steps. The extension reaction may be performed in the presence of non-standard nucleotides, and/or in the presence of non-standard nucleotides conjugated to a reporter molecule or detectable label such as biotin. Thus, the detection step may include detecting the detectable label by methods known in the art (e.g., exposing the biotin to fluorescent streptavidin-phycoerythrin (“SAPE”) and reading the fluorescent signals). Additionally or alternatively, the TSE primer may include one or more non-standard bases such as iso-C and iso-G; a non-standard base may be included in the tag region or in the viral specific region or both.

An embodiment is illustrated in FIG. 3. In FIG. 3, two or more groups of capture oligonucleotides 202 are prepared. Each group of capture oligonucleotides 202 includes a unique molecular recognition sequence 204. The molecular recognition sequence of each group includes at least one (and, typically, two or more) non-standard bases (denoted by the use of dashed lines in the figures). The use of non-standard bases substantially reduces the likelihood that the capture oligonucleotides will hybridize with sequences that include only natural bases. This will typically result in less non-specific hybridization when compared to a similar assay using oligonucleotides with only natural bases. The capture oligonucleotide also typically includes a reactive functional group for attachment to the solid support 206, although other attachment methods can be used, as described above.

The target oligonucleotide 208, if present in the assayed sample, contains an analyte-specific sequence 210 and a tagging sequence 212 complementary to the molecular recognition sequence 204 of one group of the capture oligonucleotides 202. The tagging sequence 212 contains at least one non-standard base; otherwise the tagging sequence would not be complementary to the molecular recognition sequence of the capture oligonucleotide. An oligonucleotide 214 complementary to a portion of the target oligonucleotide 208 includes a reporter 216 or a coupling agent (not shown) for attachment of a reporter.

The target oligonucleotide 208 and complementary oligonucleotide 214 can be formed by, for example, PCR amplification of an analyte containing the analyte-specific sequence or its complement. In PCR amplification, two different primers are used (as illustrated at B of FIG. 3). A first primer 218 contains a sequence complementary to a first sequence on a first strand of the analyte 220. A second primer 222 contains a sequence that is the complementary to a second sequence on a second strand of the analyte 220 which is upstream or downstream of the first sequence. The analyte-specific sequence typically includes the sequence of the analyte stretching between, and including, the sequences (or complements) to which the primers hybridize. The first primer 218 includes the tagging sequence 212 and the second primer 222 includes the reporter 216 (or a coupling agent for a reporter). Extension of the first and second primers and subsequent PCR amplification produces the target oligonucleotide 208 and complementary oligonucleotide 214 (as illustrated at C of FIG. 3). Other known synthetic methods, such as, for example, solid state synthesis, DNA replication, reverse transcription and the like, can be used to form the target and complementary oligonucleotides.

The target oligonucleotide 208 is then brought into contact with the support 206 (or a container holding a particulate support) with associated capture oligonucleotides 202 such that the capture oligonucleotide and the target oligonucleotide selectively hybridize (as illustrated at D of FIG. 3). A reporter is also added (unless the complementary oligonucleotide 214 already contains the reporter) for coupling to the complementary oligonucleotide 214.

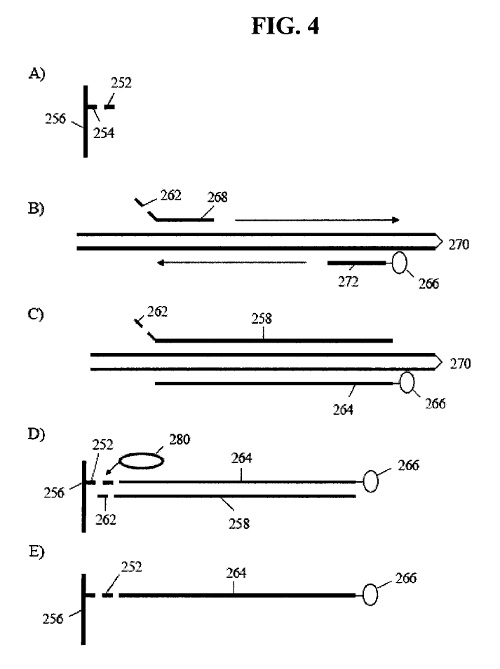

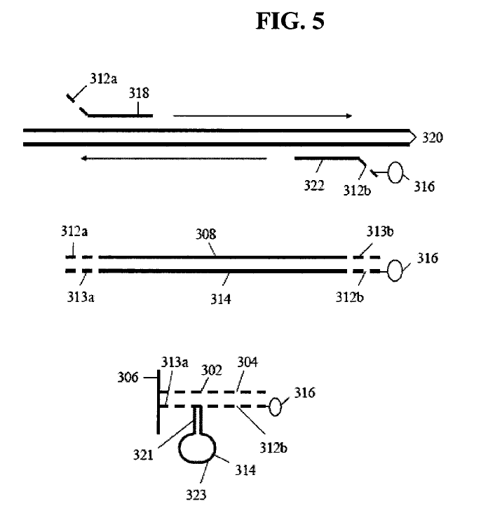

In another assay illustrated in FIG. 4, two or more groups of capture oligonucleotides 252 are prepared and placed on a support 256, as illustrated at A of FIG. 5. Each group of capture oligonucleotides 252 includes a unique molecular recognition sequence 254. The molecular recognition sequence of each group includes at least one (and, typically, two or more) non-standard bases. A target oligonucleotide 258 and complementary oligonucleotide 264 can be formed by, for example, PCR amplification of an analyte containing the analyte-specific sequence or its complement. In PCR amplification, two different primers are used (as illustrated at B and C of FIG. 4). A first primer 268 contains a sequence complementary to a first sequence on a first strand of the analyte 270. A second primer 272 contains a sequence that is the complementary to a second sequence on a second strand of the analyte 270 which is upstream or downstream of the first sequence. The analyte-specific sequence typically includes the sequence of the analyte stretching between, and including, the sequences (or complements) to which the primers hybridize. The first primer 268 includes the tagging sequence 262 and the second primer 272 includes the reporter 266 (or a coupling agent for a reporter).

The target oligonucleotide 258 is then brought into contact with the support 256 (or a container holding a particulate support) with associated capture oligonucleotides 252 so that the capture oligonucleotide and the target can selectively hybridize (as illustrated at D of FIG. 4). A reporter is also added (unless the complementary oligonucleotide 264 already contains the reporter) for coupling to the complementary oligonucleotide 264.

An enzyme 280 is then provided to covalently couple the complementary oligonucleotide 264 to the capture oligonucleotide 252. Suitable enzymes include ligases. Optionally, the target oligonucleotide 258 is denatured from the complementary oligonucleotide 264 and the target oligonucleotide and other components of the assay are washed away leaving the complementary oligonucleotide 264 bound to the support 256, as illustrated at E of FIG. 4. The reporter 266 can then be detected.

In yet another assay embodiment illustrated in FIG. 5, the target oligonucleotide 314 forms a hairpin or stem-loop structure 321, 323 (or structure other than the typical double helix). In this assay, each of the first and second primers 318, 322 includes a portion of the tagging sequence 312b or a complement to a portion of the tagging sequence 312a. In addition, one of the primers 322 has a reporter 316 (or coupling agent for a reporter) attached to the portion of the tagging sequence 312b. Using, for example, PCR techniques, the first and second primers 318, 322 amplify the analyte 320 to produce a target oligonucleotide 314 and its complement 308. The tagging sequence 312b, 313a of the target oligonucleotide 314 is distributed at both ends of the target oligonucleotide.

The target oligonucleotide 314 is denatured from its complement 308 and brought into contact with the solid support 306 having capture oligonucleotides 302 with molecular recognition sequences 304. If the molecular recognition sequence 304 of one of the capture oligonucleotides is complementary to the tagging sequence 312b, 313a of the target oligonucleotide 314, the target oligonucleotide 314 will hybridize to that capture oligonucleotide. In some embodiments, the capture oligonucleotide is divided into two parts, each part complementary with one of the parts of the tagging sequence 312b, 313a. The two parts are coupled by a linker. The linker can be additional nucleotides or any other chemical linking moiety. The target sequence of the target oligonucleotide 314 forms at least part of a stem-loop structure 321, 323 (or structure other than an double helix). Detection is then performed as discussed above in the previous examples.

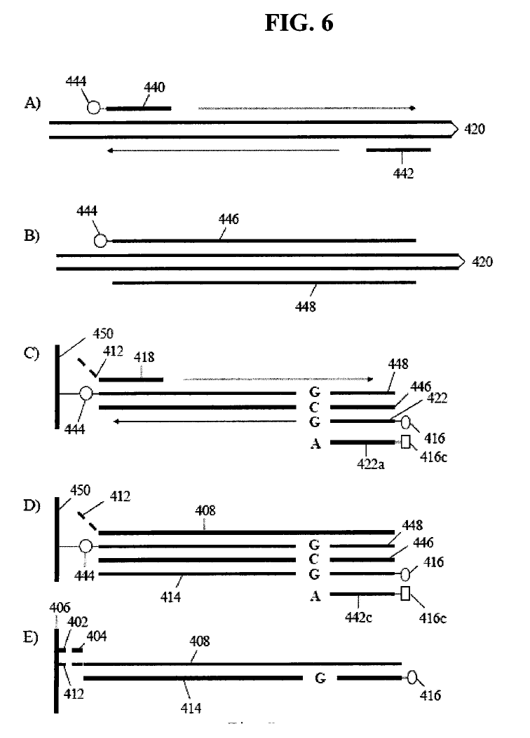

In an alternative assay illustrated in FIG. 6, an analyte 420 is contacted by initial primers 440, 442 each having a sequence that is complementary to a sequence of the analyte 420, as illustrated at A of FIG. 6. One of the initial primers 440 also includes a coupling group 444 (e.g., biotin or a substituent containing a reactive functionality) for attachment to a substrate 450. The initial primers 440, 442 are extended using, for example, PCR techniques, as illustrated at B of FIG. 6. The extended initial primers 446, 448 each include the analyte-specific sequence or its complement.

The extended initial primers 446, 448 are then brought into contact with a substrate 450 that interacts with the coupling group 444 of extended initial primer 446 to attach the extended initial primer 446 to the substrate 450, as illustrated at C of FIG. 6. For example, the substrate can be coated with streptavidin and the extended initial primer include biotin.

Next, first and second primers 418, 422 are brought into contact with the extended initial primers 446, 448, as illustrated at C of FIG. 6. The first primer 418 has a tagging sequence 412 and the second primer 422 has a reporter 416 (or coupling agent for a reporter). Both primers also include a sequence complementary to a section of the extended initial primers 446, 448.

In this illustrated assay, primers 422, 422a are “allele-specific” primers with “allele-specific” reporters 416, 416a. In the illustrated example, the alleles differ by a single nucleotide, although it will be understood that other allele-specific assays with more than one nucleotide difference can be performed using these techniques. Such primers may be used, for example, to further characterize viral sub-types, mutants or variants of interest within a particular viral group. Primer 422 is extended because it is complementary to a sequence on the extended initial primer 446. Primer 422a does not extend because it is not complementary to extended initial primer 446. It will be recognized that an alternative assay includes several different allele-specific primers with allele-specific tagging sequences (as opposed to allele-specific reporters). It will also be recognized that another alternative assay includes non-allelic primers for determination of the presence of absence of non-allelic analyte-specific sequences in the analyte.

The primers 418, 422 are extended to form the target oligonucleotide 408 with the tagging sequence 412 and the complementary oligonucleotide 414 with the reporter 416 (or a coupling agent for a reporter). The target oligonucleotide 408 and complementary oligonucleotide 414 are denatured from the extended initial primers 446, 448 and brought into contact with capture oligonucleotides 402 on a solid support 406 (e.g., chip, wafer, or particles). The target oligonucleotide 414 hybridizes to a capture oligonucleotide 402 having a molecular recognition sequence 404 complementary to the tagging sequence 412. The presence or absence of particular analyte-specific sequences in the analyte is determined by observation of the presence or absence of reporter associated with each unique group of capture oligonucleotides.

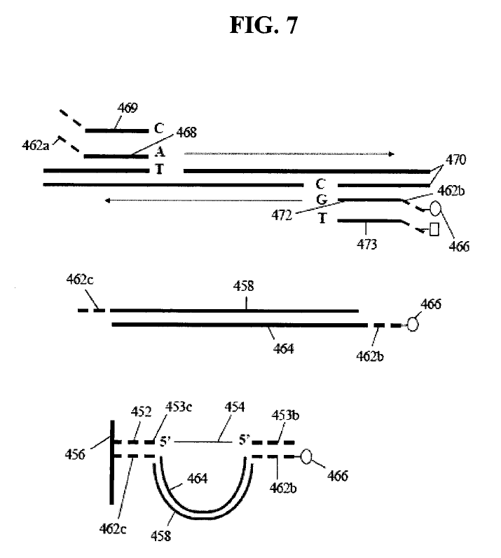

In another example of an assay illustrated in FIG. 7, a first primer 468 and a second primer 472 are brought into contact with an analyte 470 and extended to form a target oligonucleotide 458 and complementary oligonucleotide 464. In the illustrated example, the first and second primers 468, 472 are both allele-specific, but specific to different alleles. In addition to the first and second primers 468, 472, other first and second primers 469, 473 are included to amplify other alleles, if present in the sample.

The first primer 468 includes a first part 462a of a tagging sequence and the second primer 472 includes a second part 462b of the tagging sequence. One of the parts 462a, 462b includes a reporter 466 (or coupling agent for a reporter). Typically, the parts 462a, 462b of the tagging sequence will be configured so that the extension of the primers 468, 472 does not proceed through the tagging sequence. For example, the parts 462a, 462b can include a non-standard base as the base linking the part of the tagging sequence to the extendable portion of the primers 468, 472. In this embodiment, the nucleotide triphosphate of the complement of the non-standard base is not included in the PCR amplification process. Alternatively, a chemical linker can be used to couple the part of the tagging sequence to the extendable portion of the primer. Examples of suitable linkers include, but are not limited to, n-propyl, triethylene glycol, hexaethylene glycol, 1′, 2′ dideoxyribose, 2′-O-methylriboneucleotides, deoxyisocytidine, or any linkage that would halt the polymerase.

A coupling oligonucleotide 452 is provided on a support 456. The coupling oligonucleotide 452 includes parts 453a, 453b that are complementary to the parts 462a, 462b of the tagging sequence. These parts 453a, 453b are coupled by a chemical or nucleotidic linker 454 that is capable of coupling 5′ (or 3′) ends of two nucleotidic sequences.

The target oligonucleotide 458 and complementary oligonucleotide 464 are brought in contact with the support 456 and capture oligonucleotide 452 to hybridize the corresponding parts 453a, 453b of the capture oligonucleotide with the respective parts 462a, 462b of the tagging sequence. The remainder of the target oligonucleotide 458 and complementary oligonucleotide 464 will typically form a structure such as that illustrated in FIG. 7.

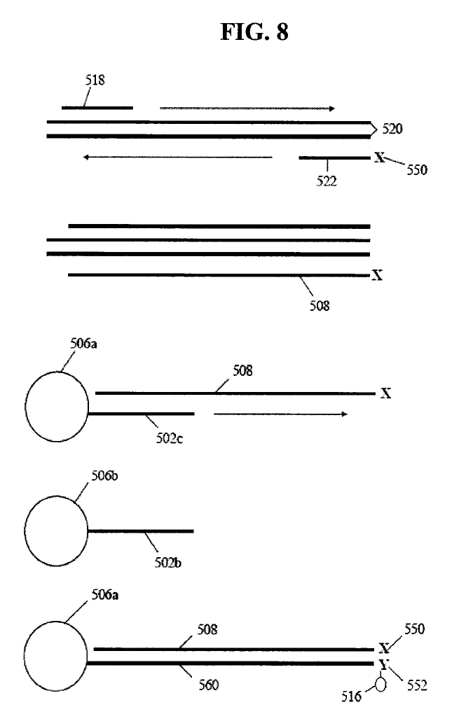

FIG. 8 illustrates one type of assay which includes the incorporation of a non-standard base by PCR. First and second primers 518, 522 are hybridized to analyte 520 and extended. One of the primers 522 includes a non-standard base 550 which, when extended, becomes the target oligonucleotide 508. Optionally, additional bases can be provided after the non-standard base 550. The target oligonucleotide 508 with the non-standard base 550 is then brought into contact with the solid support 506a, 506b that includes capture oligonucleotides 502a, 502b. The solid support illustrated in FIG. 8 is the particulate support discussed above, however, it will be recognized that a single solid support (e.g., a chip or wafer) could also be used.

The capture oligonucleotides 502a, 502b are different and are attached to different supports 506a, 506b, respectively, so that the capture oligonucleotide can be recognized by observing the unique property of the support to which it is attached. One capture oligonucleotide 502a hybridizes with the target oligonucleotide 508. The capture oligonucleotide 502a in this embodiment has a sequence that is complementary to at least a portion of the analyte-specific sequence of the target oligonucleotide 508.

After hybridization of the target oligonucleotide 508, the capture oligonucleotide 502a is extended in a PCR solution that includes dATP, dUTP, dGTP, dCTP, and the nucleotide triphosphate of a second non-standard base (e.g., diso-GTP) 552 complementary to the non-standard base 550 on the target oligonucleotide 508. The second non-standard base 552 is labeled with a reporter 516 (or coupling agent for a reporter). As the capture oligonucleotide is extended, the second non-standard base 552 with the reporter 516 is incorporated into the extended capture oligonucleotide opposite the non-standard base 550. Thus, the presence or absence of a reporter on a particular group of particulate supports indicates the presence or absence of a particular target oligonucleotide associated with the capture oligonucleotide.

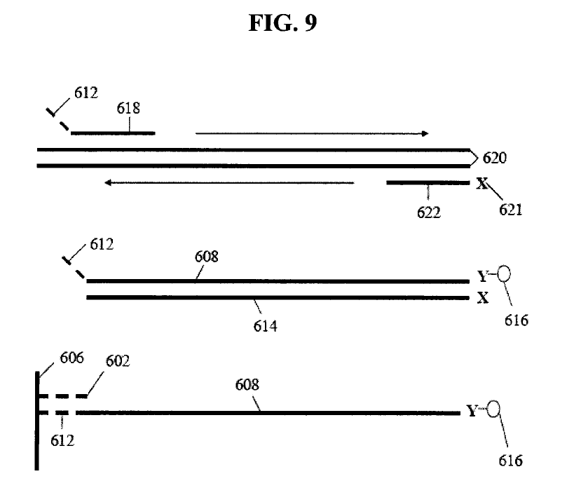

FIG. 9 illustrates another assay. In this assay, the first primer 618 includes a tagging sequence 612 and the second primer 622 has a non-standard base 621 (or a sequence containing a non-standard base) at its 5′ end. The primers 618, 622 amplify the analyte 620 in the presence of the dATP, dCTP, dGTP, dTTP, and the nucleotide triphosphate of the non-standard base complementary to non-standard base 621. This non-standard base nucleotide triphosphate is labeled with a reporter 616 (or coupling group for a reporter) and is incorporated opposite non-standard base 621 to form the target oligonucleotide 608.

The target oligonucleotide 608 is brought into contact with the solid support 606 having capture oligonucleotides 602 with molecular recognition sequences. If one of the molecular recognition sequences is complementary to the tagging sequence 612 of the target oligonucleotide 608, the target oligonucleotide 608 will hybridize to the capture oligonucleotide 602. Detection is then performed as discussed above in the previous examples.

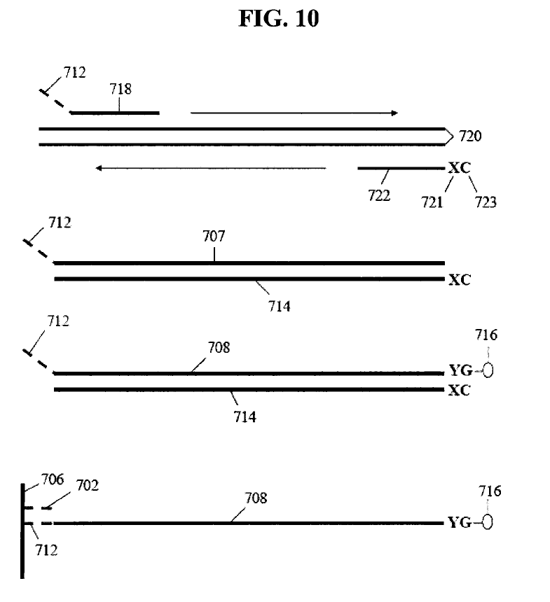

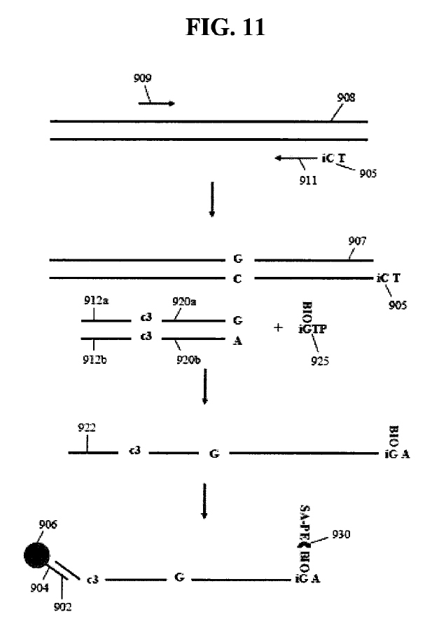

FIG. 10 illustrates yet another assay. In this assay, the first primer 718 includes a tagging sequence 712 and the second primer 722 has a non-standard base 721 followed by a natural base 723 (or a sequence of natural bases) at its 5′ end. The primers 718, 722 amplify the analyte 720 in the presence of the dATP, dCTP, dGTP, and dTTP only to form a partially extended target oligonucleotide 707 and its complement 714. The extension of the partially extended target oligonucleotide is limited by the non-standard base 721. After this initial amplification, the amplification products 707, 714 are washed to remove dATP, dCTP, dGTP, and dTTP.

A second extension step is then performed, in the presence of the triphosphate of the non-standard base complementary to non-standard base 721 and at least the triphosphate of the natural base complementary to natural base 723. This natural base triphosphate is labeled with a reporter 716 (or coupling group for a reporter) and is incorporated opposite natural base 723 to form the target oligonucleotide 708.

The target oligonucleotide 708 is brought into contact with the solid support 706 having capture oligonucleotides 702 with molecular recognition sequences. If one of the molecular recognition sequences is complementary to the tagging sequence 712 of the target oligonucleotide 708, the target oligonucleotide 708 will hybridize to the capture oligonucleotide 702. Detection is then performed as discussed above in the previous examples.

In one embodiment, allele-specific second primers may be used with the same first primer. In this example, the allele-specific second primers are differentiated in the portion of the second primer that anneals to the analyte. A different natural base 723 is selected for each allele. During the second extension step, where bases are added opposite the non-standard base 721 and natural base 723, the nucleotide triphosphates of two or more natural bases are added to the extension mixture. The different nucleotide triphosphates are labeled with different reporters. Thus, if the natural base 723 can be A or C, depending on the allele, the dTTP and dGTP used in the extension step are labeled with different reporters. The identity of the reporter can be used to determine the presence of a particular, associated allele. Thus, for example, four different alleles can be simultaneously tested using this method and, with appropriate choice of reporters, can be indicated using four different colors.

In an assay embodiment illustrated in FIG. 10, two or more groups of capture oligonucleotides 902 are prepared. Each group of capture oligonucleotides 902 includes a unique molecular recognition sequence 904. The molecular recognition sequence of each group optionally includes at least one or more non-standard bases. The capture oligonucleotide also typically includes a reactive functional group for attachment to a solid support 906, although other attachment methods can be used, as described above.

The target oligonucleotide 908, if present in the assayed sample, is contacted with a first primer 909 and a second primer 911. The first and second primers 909, 911 can be allele-specific or, preferably, are not complementary to allele specific portions of the target oligonucleotide (i.e., the allele specific portions of interest are positioned within the target oligonucleotide between the regions that hybridize to the two primers). The second primer 911 also includes a non-complementary attachment region 905. This non-complementary reporter attachment region 905 optionally includes one or more non-standard bases. The target oligonucleotide 908 is amplified using the first and second primers 909, 911 and PCR techniques to obtain an amplification product 907 that includes the reporter attachment region 905.

The amplification product 907 is then contacted with allele specific primers 920a, 920b that are then extended, if the particular allele is present, using reaction conditions and reaction components similar to PCR to provide an allele specific extension product 922. Each allele specific primer 920a, 920b has an allele-specific tagging sequence 912a, 912b that is complementary to different molecular recognition sequences 904 and capture oligonucleotides 902. When extending the allele specific primers 920a, 920b, a labeled nucleotide 925 (or oligonucleotide) that is complementary to one or more bases of the attachment region 905 is provided. The labeled nucleotide 925 or oligonucleotide can include a reporter or a coupling agent, such as biotin, for attachment of a reporter.

After forming the extension product 922, contact is made with the capture oligonucleotides 902 and with a reporter 930 (unless a reporter was already attached). The capture oligonucleotide 902 and the support 906 identify which allele(s) is/are present in the sample and the reporter provides for detection of the extension product 922. For assays on particle supports, the particles can be separated according to the unique characteristics and then it is determined which particles 906 have a reporter coupled to the particle via the capture oligonucleotide 902 and extension product 922. Techniques for accomplishing the separation include, for example, flow cytometry. The presence of the reporter group indicates that the sample contains the allele associated with a particular allele-specific tagging sequence.

### Assay Components and Methods

Solids Supports. In general, an assay system for detecting one or more analyte-specific sequences includes a solid support (e.g., a chip, wafer, the interior or exterior of a tube, cone or other article, or a collection of solid particles). Capture oligonucleotides may be coupled to or otherwise disposed on the solid support in a manner which permits identification of the capture oligonucleotide (e.g., by position on a chip or wafer or by unique characteristic of particles to which particular capture oligonucleotides are attached, for example color addressed microspheres). Materials and methods used to couple capture oligonucleotides to solid supports are well known in the art.

A variety of different supports can be used. In some embodiments, the solid support may be a single solid support, such as a microscope slide, chip or wafer, or the interior or exterior surface of a tube, cone, or other article. Materials and methods to generate such supports are well known in the art; however, in some embodiments, preferred materials may include polystyrene, glass, and silicon.

In other embodiments, the solid support may be a particulate support. In these embodiments, the capture oligonucleotides are coupled to particles. The particles may form groups in which particles within each group have a particular characteristic, such as, for example, color, fluorescence frequency, density, size, or shape, which can be used to distinguish or separate those particles from particles of other groups. In some embodiments, the particles may be separated using techniques, such as, for example, flow cytometry. The particles may be fabricated from virtually any insoluble or solid material; such materials and methods are well known in the art. By way of example, but not by way of limitation, micro-beads are described in U.S. Pat. Nos. 5,736,330, 6,046,807, and 6,057,107, all of which are incorporated herein by reference, and particles are available, for example, from Luminex Corp., Austin, Tex.

In still other embodiments, the support may be a group of individual support surfaces that are optionally coupled together. For example, the support may include individual optical fibers or other support members that are individually coupled to different capture oligonucleotides and then bound together to form a single article, such as a matrix.

In some embodiments, a single solid support may be divided into individual regions with different capture oligonucleotides disposed on the support in each region. For example, an array can be formed to test for 10, 100, 1000 or more different analyte-specific sequences. Similarly, different groups of particle supports may include different capture oligonucleotides. In each of the regions or on each particle support, the capture oligonucleotides may have predominantly (e.g., at least 75%) the same molecular recognition sequence. In other embodiments, the capture oligonucleotides may have substantially all (e.g., at least 90% or at least 99%) the same molecular recognition sequence in each region or on each particle support. The capture oligonucleotides of different regions typically have different sequences, although in some instances, the same capture oligonucleotides can be used in two or more regions, for example, as a control or verification of results.

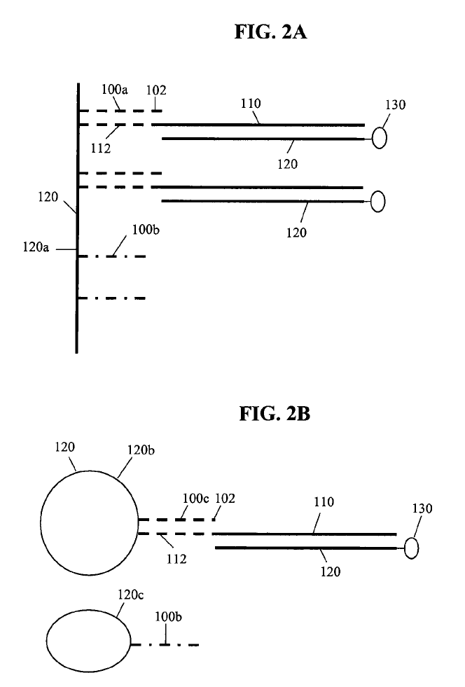

Capture Oligonucleotides and Target Oligonucleotides. Exemplary capture systems are schematically illustrated in FIGS. 2A and 2B. In these examples, capture oligonucleotides 100a, 100b are coupled to a solid support 120, such as, for example, a single solid substrate 120a (e.g., a chip or wafer) or one of a number of solid particles 120b. Typically, at least one of the capture oligonucleotides (e.g., capture oligonucleotide 100a) has a molecular recognition sequence 102 that is complementary to a tagging sequence 112 of a target oligonucleotide 110 so that, under hybridization conditions, the target oligonucleotide 110 hybridizes to the capture oligonucleotide 100a.

Although assays can be prepared with all of the capture oligonucleotides having the same global molecular recognition sequence, typically, two or more different groups of capture oligonucleotides 100a, 100b are used. Each group of capture oligonucleotides has a different molecular recognition sequence. On a single solid substrate, each group of capture oligonucleotides are typically disposed on a particular region or regions of the substrate such that the region(s) is/are associated with a particular molecular recognition sequence. When a particle support is used, each group of capture oligonucleotides 100a, 100b may be disposed on at least one group of particles 120b, 120c having a unique characteristic such that the capture oligonucleotide of a particular particle is determined from the characteristic of the particle to which it is attached. Such assays can be used to assay for multiple viral groups, serotypes, etc. As illustrated in FIGS. 2A and 2B, the target oligonucleotide preferentially hybridizes to a corresponding capture oligonucleotide permitting determination of the presence or absence of an analyte-specific sequence by observation of the presence or absence of a target oligonucleotide on a particular spatial position of the single support (FIG. 2A) or attached to a particular group of particles (FIG. 2B). A reporter 130 that couples to the target oligonucleotide 110 (or its complement 120) is added and subsequently detected.

The capture oligonucleotide includes a molecular recognition sequence that can capture, by hybridization, a target oligonucleotide having a complementary tagging and/or a complementary analyte specific sequence. The hybridization of the molecular recognition sequence of a capture oligonucleotide and the complementary sequence of a target oligonucleotide results in the coupling of the target oligonucleotide to the solid support. The molecular recognition sequence is thus associated with a particular analyte-specific sequence (also part of the target oligonucleotide), thus indicating, if hybridization occurs, the presence or concentration of analyte with the analyte-specific sequence (or its complement) in the original sample. The molecular recognition and tagging sequences may include at least six nucleotides and, in some instances, include at least 8, 10, 15, or 20 or more nucleotides. In some assays, the molecular recognition sequence and tagging sequence include one or more non-standard bases. In other assays, the molecular recognition sequence and tagging sequence do not contain non-standard bases.

In some embodiments, the different molecular recognition sequences of the capture oligonucleotides are not complementary to one another and, more preferably, to any known natural gene sequence or gene fragment that has a significant probability of being present in a substantial amount in the sample to be tested. As a result, the molecular recognition sequences of the capture oligonucleotides can primarily hybridize to the respective complementary tagging sequences of the target oligonucleotides.

Selection of Molecular Recognition Sequences. When multiple molecular recognition sequences are used to form an assay system that can detect more than one analyte-specific sequence with the application of a single sample, a collection of different molecular recognition sequences is typically needed. Preferably, the molecular recognition sequences are sufficiently different to permit reliable detection of analyte-specific sequences under a desired set of stringency conditions. A variety of different methods can be used to choose the collection of molecular recognition sequences. The following is a description of some methods and criteria that can be used. The methods and criteria can be used individually or in combinations.

The following are examples of criteria that can be used in creating a collection of molecular recognition sequences: the number of bases in the sequence, the number of non-standard bases in the sequence, the number of consecutive natural bases in the sequence, the number of consecutive bases (in either the forward or reverse directions) that are the same in any two sequences, specific required sequences (e.g., GC clamps at the 3′ or 5′ ends or both) and the estimated or actual melting temperature. One example of a method for determining Tm is described in Peyret et al., Biochemistry, 38, 3468-77 (1999), incorporated herein by reference. The non-standard bases can be estimated or accounted for using, for example, values for other bases (e.g., iso-G/iso-C can be estimated using G/C) or using experimental data such as that described below.

The following are a set of steps that can be used to form the collection of molecular recognition sequences: (1) Create a set of all possible oligonucleotides having a length of n1 (e.g., 8, 9, or 10 nucleotides) using the natural bases and the desired non-standard bases (e.g., iso-C, iso-G, or both); (2) Optionally require that the oligonucleotides have a particular subsequence (e.g., GC clamps on the 3′ or 5′ ends or both ends); (3) Remove oligonucleotides without at least n2 non-standard bases (e.g., without at least two iso-C bases) or with more than n3 non-standard bases (e.g., with more than two iso-C bases) or both (e.g., accept only oligonucleotides with exactly two iso-C bases); (4) Optionally remove oligonucleotides with n4 (e.g., four or five) natural bases in a row; (5) Select one of the remaining oligonucleotides and eliminate any of the remaining oligonucleotides that have n5 bases (e.g., five or six bases) in the same order anywhere in the oligonucleotide sequence. Repeat for each non-eliminated oligonucleotide; (6) Optionally select one of the remaining oligonucleotides and determine its reverse complement (e.g., the reverse complement of “ACT” is “AGT”), then eliminate any of the other oligonucleotides that have n6 consecutive bases (e.g., four or five bases) that are the same as a portion of the sequence of the reverse complement. Repeat for each non-eliminated oligonucleotide; and (7) Optionally select only the remaining oligonucleotides that have an estimated or actual melting temperature (Tm) within a desired temperature range, above a desired temperature limit, or below a desired temperature limit. For example, oligonucleotides can be eliminated that having a melting temperature below room temperature (about 22° C.).

The length of the capture oligonucleotides can be optimized for desired hybridization strength and kinetics. In some embodiments, the length of the molecular recognition sequence is in the 6 to 20 (preferably, 8 to 12) nucleotide range.

The capture oligonucleotide may also include a functional group for example, to permit the binding of the capture oligonucleotide to the solid support.

The target oligonucleotide (or an oligonucleotide complementary to at least a portion of the target oligonucleotide) may include a reporter or a coupling agent for attachment of a reporter. The reporter or coupling agent can be attached to the polymeric backbone or any of the bases of the target or complementary oligonucleotide. Techniques are known for attaching a reporter group to nucleotide bases (both natural and non-standard bases). Examples of reporter groups include biotin, digoxigenin, spin-label groups, radio labels, DNA-cleaving moieties, chromaphores, and fluorophores such as fluoroscein. Examples of coupling agents include biotin or substituents containing reactive functional groups. The reporter group is then attached to streptavidin or contains a reactive functional group that interacts with the coupling agent to bind the reporter group to the target or complementary oligonucleotide.

In addition to the tagging sequence, the target oligonucleotide includes an analyte-specific sequence which corresponds to or is a complement to a sequence of interest in the analyte. The analyte-specific sequence can be independent from the tagging sequence or some or all of the tagging sequence can be part of the analyte-specific sequence.

Capture Methods. In some embodiments, the particulate supports with associated capture oligonucleotides may be disposed in a holder, such as, for example, a vial, tube, or well. The target oligonucleotide may then be added to the holder and the assay may be conducted under hybridization conditions. In some embodiments, multiple holders (e.g., vials, tubes, and the like) are used to assay multiple samples or have different combinations of capture oligonucleotides (and associated supports) within each holder. As another alternative, each holder can include a single type of capture oligonucleotide (and associated support). Additionally or alternatively, prior to contact with the support(s) and capture oligonucleotides, the solution containing target oligonucleotides can be subjected to, for example, size exclusion chromatography, differential precipitation, spin columns, or filter columns to remove primers that have not been amplified or to remove other materials that are not the same size as the target oligonucleotides.

Conditions are controlled to promote selective hybridization of the tagging sequence of the target oligonucleotide with a complementary molecular recognition sequence of a capture oligonucleotide, if an appropriate capture oligonucleotide is present on the support. A reporter may also be added (unless the target or its complement already include a reporter).

The particulate supports may then be separated on the basis of the unique characteristics of each group of supports. The groups of supports are then investigated to determine which support(s) have attached target oligonucleotides. Optionally, the supports can be washed to reduce the effects of cross-hybridization. One or more washes can be performed at the same or different levels of stringency.

Detection Methods. For assays on a planar solid support, the assay may be read, for example, by determining whether the reporter group is present at each of the individual regions on the support. The presence of the reporter group indicates that the original sample contains an analyte having the analyte-specific sequence associated with the particular tagging sequence and molecular recognition sequence for that region of the support. The absence of the reporter group suggests that the sample did not contain an analyte having the particular analyte-specific sequence.

For assays on particle supports, the particles may be separated according to the unique characteristics and then it may determined which particles have a reporter coupled to the particle via the capture and target oligonucleotides. Techniques for accomplishing the separation include, for example, flow cytometry. The presence of the reporter group indicates that the sample contains the target oligonucleotide having the analyte-specific sequence associated with a particular tagging sequence and the molecular recognition sequence of a particular capture oligonucleotide.

### EXAMPLES

The following examples are provided to aid the reader to better understand the methods and compositions described herein and are meant to be non-limiting. One skilled in the art would understand that in many instances, different methods, reagents, conditions, etc. may be used to achieve the same or similar results.

Generally, a patient sample containing an analyte (e.g., viral sequence) of interest is subject to RT-PCR (e.g., if an RNA virus) to generate cDNA. The cDNA is then amplified by PCR using primers specific for the analyte specific sequence to generate amplicons. At least one of the PCR primers includes at least one non-standard base. Further, the amplification may be performed in the presence of non-standard nucleotides, thereby allowing incorporation of base-paired, non-standard nucleotides in the amplicons. The amplicons are then tagged by introducing at least one TSE primer. The TSE primer includes, minimally, a 3′ region specific to the analyte-specific sequence (e.g., viral-specific sequence) and a 5′ tagging sequence which does not hybridize to the analyte sequence. Extension with the TSE primer produces tagged target oligonucleotides; the tagging sequence is used in the “capture” step described below.

Extension with the TSE primer may be performed in the presence of a non-standard nucleotide conjugated to a detectable label or reporter molecule such as biotin. In some embodiments, the label or reporter may be coupled to a non-standard nucleotide which can be incorporated into the TSE extension product across from the non-standard nucleotide of the amplicon. Thus, resulting target oligonucleotides will include both a tagging sequence and a detectable label.

Additionally or alternatively, the TSE primer may include at least one detectable label, such as biotin, and may also include one or more non-standard nucleotides. If a non-standard base is present in the tagging sequence, the capture oligonucleotide may also include a complementary non-standard base. If the non-standard base is in the analyte-specific region, the PCR primers may be designed such that the non-standard base of the TSE primer will hybridize to a non-standard base of the amplicon.

Capture oligonucleotides, designed to include a sequence complementary to the tagging sequence (the molecular recognition sequence), are coupled to a solid support, such as a microsphere. These microspheres are contacted with the target oligonucleotides, and hybridization between the capture oligonucleotide and the target oligonucleotide allows for the “capture” of the target oligonucleotide. The reporter may then be detected by methods known in the art, and the target oligonucleotides may then be identified and quantitated. Flow cytometry is an exemplary detection method.

### Example 1

### Identification of Detection Target Sequences for Common Respiratory Viruses

For the following examples, 8 respiratory virus groups (HRV, RSV, InfV, PIV, MPV, AdV, CoV, and EnV) with multiple strains/serotypes were selected for analysis. Eighteen sets of virus-specific multiplex-PCR primers were developed based on the conserved sequences of all available respiratory viral sequences for the eight groups. It is understood that the methods described herein can be applied to other virus groups and/or other serotypes.

To be able to detect all members of each virus group, conserved viral genomic regions were identified as the detection targets for designing MultiCode PCR primers. First, all full-length genome sequences of each virus group (see Table 1, below, total of 110 sequences) listed in public databases, including NCBI Taxonomy Brower, NCBI nucleotide database, Picornavirus Home Page and the Influenza Sequence Database, were analyzed with the alignment program ClustalX (Thompson, J. D., et al. 1997. Nucleic Acids Research 24:4876-4882.).

HRV has 101 known serotypes, but only 7 full-length sequences (serotypes 1B, 2, 9, 14, 16, 85 and 89) were found in the databases. Alignment of these 7 sequences showed that the 5′ noncoding region (NCR) was the most conserved genomic region, and it was therefore chosen as the detection target.

The EnV group includes polioviruses (3 serotypes), echoviruses (29 serotypes), coxsakieviruses (29 serotypes) and EV68 through EV73. Polioviruses and echoviruses were excluded from our target list because they are not considered as respiratory pathogens. Sixty-two full-length sequences for coxsackieviruses and EV68 through EV73 were identified. Like HRV, the 5′ NCR was selected as the detection target.

Coronaviruses (CoV) have 3 serotypes that are common respiratory pathogens: OC43, 229E and NL63, although additional serotypes have been recently identified. Alignment of the 2 full-length genome sequences of OC43 and 229E and 3 sequences of NL63 showed very little homology, and separate primer sets were designed for each serotype. The nucleocapsid (N) gene was chosen as the detection target for coronaviruses because it is highly expressed in infected cells.

RSV have 2 serotypes: A and B, and 10 full-length genome sequences (7 RSVA and 3 RSVB) were identified. Although the fusion (F) gene was the most conserved, variability led to synthesis of separate primer sets for each serotype.

For metapneumoviruses (MPV), 4 full-length sequences were found, and the polymerase (L) gene or the fusion gene were selected as the detection target.

Parainfluenza viruses (PIV) have 5 serotypes: 1, 2, 3, 4a and 4b. Full-length sequences were found for PIV1 (2 sequences), PIV2 (n=2) and PIV3 (n=2) but not for PIV4a and 4b. Although there was some sequence homology in the hemagglutination-neuraminidase (HN) gene, serotype-specific primer sets were developed instead.

Influenza viruses (InfV) has 3 genera: A, B and C. InfV C was not included in the assay because it is not a common pathogen. Unlike the other virus groups, the genomes of InfV A and B are divided into 8 RNA strands. Only 2 of the most conserved segments were examined: matrix (M) and nucleoprotein (NP). Analysis of 51 NP (28 A and 23 B) and 59 M (40 A and 19 B) sequences of the recent (after 1990) human isolates showed that M genes were slightly more conserved than NP genes, while M and NP sequences varied between InfV A and B. Therefore, M gene sequences were chosen as the detection target and separate primer sets were selected for InfV A and B.

Adenoviruses (AdV) have more than 50 serotypes that are divided into 6 groups (A to F) based on their ability to agglutinate red blood cells, and group B, C and E viruses were selected for our assay because they are common respiratory pathogens. Fourteen full-length sequences were found in GenBank: 7 B, 6 C and 1 E. Alignment of these sequences showed that hexon gene was the most conserved among serotypes within the same group. Due to variability among serotypes, separate primer sets were needed for each group.

Collectively, based on the alignment analysis of all available complete genome sequences, 18 conserved viral genomic regions were identified as the detection targets for the 8 respiratory virus groups (Table 1). The sizes of these target viral genomic regions, except for the L gene of MPV, occupy about 10% or less of the respective genomes (Table 1).

339 additional partial sequences (Table 1) were identified in public databases and were included in the primer design process to maximize the sensitivity of the assay. Altogether, searches of public databases yielded a total of 449 sequences (110 complete genomes and 339 target genomic regions) for designing MultiCode primers.

TABLE 1
Sequence information for target respiratory viruses

| | | No. of | Target viral | Target | No. of target |
|---|---|---|---|---|---|
| Respiratory | Genome | complete | genomic | Sizes | sequences for |
| Viruses^{a} | Size (Kbs) | sequences | regions^{a} | (Kbs) | primer design |
| HRV (101 serotypes) | 7 | 7 | 5′ NCR | 0.6 | 146 |
| EnV (70 serotypes) | 8 | 62 | 5′ NCR | 0.7 | 83 |
| CoV OC43 | 30 | 2 | N gene | 1.3 | 6 |
| CoV 229E | 30 | 2 | N gene | 1.2 | 4 |
| CoV NL63 | 30 | 3 | N gene | 1.1 | 3 |
| RSV A | 15 | 7 | F gene | 1.7 | 27 |
| RSV B | 15 | 3 | F gene | 1.7 | 5 |
| MPV | 14 | 4 | L gene | 6.0 | 4 |
| PIV1 | 16 | 2 | HN gene | 1.7 | 32 |
| PIV2 | 16 | 2 | HN gene | 1.7 | 7 |
| PIV3 | 16 | 2 | HN gene | 1.7 | 14 |
| PIV4a | ^{b} | 0 | HN gene | 1.7 | 2 |
| PIV4b | ^{b} | 0 | HN gene | 1.7 | 1 |
| InfV A | 13 | ^{c} | M gene | 1.0 | 40 |
| InfV B | 13 | ^{c} | M gene | 1.0 | 19 |
| AdV B (8 serotypes) | 37 | 7 | Hexon gene | 2.8 | 35 |
| AdV C (4 serotypes) | 37 | 6 | Hexon gene | 2.9 | 16 |
| AdV E (1 serotype) | 37 | 1 | Hexon gene | 2.6 | 5 |

^{a}Abbreviations: HRV, human rhinoviruses; EnV, enteroviruses; CoV, coronaviruses; RSV, respiratory syncytial virus; MPV, metapneumoviruses; PIV, parainfluenza viruses; InfV, influenza viruses; AdV, adenoviruses; NCR = noncoding region, N = nucleocapsid, F = fusion, L = polymerase, HN = hemagglutination-neuraminidase, M = matrix.
^{b}Unknown.
^{c}The genomes InfV A and B are divided into 8 segments. Only the 2 most conserved gene segments (matrix and nucleoproteins) were analyzed.

### Example 2

### Construction of an Array of DNA Clones for Detection Targets

To generate viral targets with defined sequences and concentration for testing of MultiCode primers, the target genomic regions of the following 129 viruses (Table 2) were cloned: 5′ NCR of 101 HRV serotypes, 5′ NCR of 6 representative EnV serotypes (EV68, EV69, EV70, EV71, coxackievirus A22, coxackievirus B1), M-N genes of CoV 229E and NL63, F genes of RSV A and B, L gene of MPV, HN gene of PIV 4a and 4b, M genes of InfV A and B, and hexon genes of all 13 known serotypes of AdV B, C and E (types 3, 7, 11, 14, 16, 21, 34, 35, 1, 2, 5, 6, 4).

TABLE 2
DNA clones of target viral genomic regions

| Target viral genomic regions | Serotypes/Strains/Isolates | Size (Kb) |
|---|---|---|
| HRV 5′NCR-VP4 | HRV1-86, 88-100 and Hanks | 0.7 |
| EnV 5′NCR | EV68, 69, 70 & 71, | 0.5 |
| | CAV22 and CBV1 | |
| CoV M-N gene | 229E, NL63 and OC43 | 2.0 |
| RSV F gene | A/Long strain and B | 2.0 |
| MPV L gene | CAN97-83 | 6.0 |
| PIV HN gene | 1, 2 and 3 | 1.7 |
| | 4a and 4b | 2.5 |
| InfVA M gene | A/Sydney/05/97-like (H3N2) | 0.9 |
| InfVB M gene | B/Beijing/184/93-like | 1.0 |
| AdVB Hexon gene | 3, 7, 11, 14, 16, 21, 34 and 35 | 3.0 |
| AdVC Hexon gene | 1, 2, 5, 6 | 3.1 |
| AdVE Hexon gene | 4 | 2.8 |

Briefly, total nucleic acids including viral RNA were prepared from 100 μl of infected cell lysate by phenol extraction and ethanol precipitation. Each target viral genomic region was amplified using a reverse transcriptase (RT)-PCR mix (Invitrogen 11922-028) and appropriate primers that annealed to its 5′ and 3′ ends. (See Table 3, below). PCR products were subject to electrophoresis in a 1% low melting agarose gel. Each PCR fragment band was visualized with ethidium bromide (EtBr) staining and UV illumination in a 1% low melting agarose gel and then excised from the gel. DNA fragments were gel purified. The resulting DNA was kinased and ligated to a Stul-linearized plamsid vector pMJ3 and then transformed into E. coli. Plasmids containing the PCR fragment insertions were identified using the Colony Fast-Screen (Size) Kit (Epicentre FS08250) and agarose gel electrophoresis. Three independent plasmids for each PCR fragment were isolated, amplified and purified. The yield of each plasmid was measured optically, assuming 50 μg per unit of OD260 nm. Each viral DNA fragment was completely sequenced at the Automated DNA Sequencing Facility, University of Wisconsin Biotechnology Center.

Plasmids containing the correct viral inserts were isolated, amplified and purified. Each viral fragment was completely sequenced. Thus, this work provided not only purified viral targets with defined sequences and concentration for primer selection but also 129 new target sequences for primer design. In addition, DNA clones of the N gene of CoV OC43 and HN genes of 3 PIV serotypes (1, 2 and 3) were evaluated.

TABLE 3

Primers for RT-PCR amplification
of 129 viral detection targets

| | | |
|---|---|---|
| Viral | | |
| Targets | Amplification Primers | SEQ ID NO: |

| | | |
|---|---|---|
| PIV1: | Forward: ATGGCTGAAAAAGGGAAAACAAATAG | 99 |
| | Reverse: AGCTTAAGATGTGATTTTACATATTTTAGG | 100 |
| PIV2: | Forward: CATGGAAGATTACAGCAATCTATCTC | 101 |
| | Reverse: TTAAAGCATTAGTTCCCTTAAAAATGG | 102 |
| PIV3: | Forward: ATAGACAAATCCAAATTCGAGATGG | 103 |
| | Reverse: CTTGTATTATAGATAGATTGATGCATATTATGG | 104 |
| PIV4a: | Forward: CCCTTGCATGTGCAACC | 105 |
| | Reverse: CGATGATGTGTCGGATCG | 106 |
| PIV4b: | Forward: CACTTCTCAGCTCCAACC | 107 |
| | Reverse: GGCATGTGGAGGAACTTG | 102 |
| RSVA | Forward: CAACACAACACGCCAGTAG | 109 |
| | Reverse: TGTAAGTGAGATGGTTTATAGATAAGAG | 110 |
| RSVB | Forward: GAACACACAATCCAACAGCAATC | 111 |
| | Reverse: GGATTGGTGATCAGCAGACTG | 112 |
| InfVA: | Forward: ATGAGTCTTCTAACCGAGGTC | 113 |
| | Reverse: CTGCTGTTCCTCTCGATATTC | 114 |
| InfVB: | Forward: ATGTCGCTGTTTGGAGAC | 115 |
| | Reverse: CCCATTTTTATTATCTCTTCGGC | 116 |
| MPV-5′ | Forward: TGAATCCACTGTTAATGTCTATC | 117 |
| fragment: | Reverse: TGCTGTAGTGTATAGCACTATCAC | 118 |
| MPV-3′ | Forward: ACCAGTATCTTAAGTCTTTCCCC | 119 |
| fragment: | Reverse: TCTCTGCATTCCCTAAGTTATC | 120 |
| AdVB: | Forward: TATGGAGTAGCGCTTAACTTG | 121 |
| | Reverse: TCTTGCTCGCTGGAGCCG | 122 |
| AdVC: | Forward: AAGCACACTGAACAGCATCG | 123 |
| | Reverse: AGTTCCTGCTCACTGGAGC | 124 |
| AdVE: | Forward: CAGAAGGAGGAGTGAAGAG | 125 |
| | Reverse: CTGGTGCACTCGGACGAC | 126 |
| CoV 229E: | Forward: GTGACATTGTCACCCATTTG | 127 |
| | Reverse: CAAGGCGCCAAATCTCTC | 128 |
| CoV OC43: | Forward: ACTACACCAGCACCAGTTTA | 129 |
| | Reverse: AGATGCCGACATAAGGTTC | 130 |
| CoV NL63: | Forward: CCTGTTCCAGCTGAAGTAC | 131 |
| | Reverse: CCTTATCATGCGCTAAACG | 132 |
| EV68, 69, | Forward: CGGTACCYTTGTRCGCC | 133 |
| 70, 71, | Reverse: ATTGTCACCATWAGCAGYC | 134 |
| CAV22 and | | |
| CBV1 | | |
| HRV: | Forward: CAAGCACTTCTGTTTCCCC | 135 |
| | Reverse: | |
| | HRV1A and 1B: TCAACAGATGGTGATTGTAGTG | 136 |
| | HRV2: CCTATCAGAGTATCCACAAGC | 137 |
| | HRV3: TCTACATACAGAAGTGTCTGG | 138 |
| | HRV4: GTGTCAGGCTTGGTGG | 139 |
| | HRV5: CTGCATGCTGAAGTATCTGG | 140 |
| | HRV6: ATCTACATACAGATGTGTCTGG | 141 |
| | HRV7: GTTGAGTTGGTTTGTCAATGG | 142 |
| | HRV8: ATCTATTGGTTGAGGTATCAGG | 143 |
| | HRV9: CCATGTTTTGCTCTCCAAGG | 144 |
| | HRV10: AGTGTCAGGCTGAGTAGG | 145 |
| | HRV11: CTATTTGAGGAGGTGTCTGG | 146 |
| | HRV12: ACTCTCTAGAGTGTAAAATCGG | 147 |
| | HRV13: CCACAACTGACTTTCTAGTGTATAG | 148 |
| | HRV14: TAATTTCCAGCACCAGCC | 149 |
| | HRV15: TATCTATTGCAGTAGCATCCTC | 150 |
| | HRV16: TAGTTTCCACCACCAACC | 151 |
| | HRV17: CCATGTTTTGCTTTCTAAGGTATAA | 152 |
| | HRV18: TCTATTGGATGAAGTGTCAGG | 153 |
| | HRV19: GTAGAGGTGTCTGGTTGTG | 154 |
| | HRV20: CCTTCTTTGCTTTCCAGTG | 155 |
| | HRV21: TGAGGAAGTGTCTGGTCTAG | 156 |
| | HRV22: TGTTGGAAGATGTGTCAGG1 | 157 |
| | HRV23: AGTGTGTAGAATCTGTTGGAC | 158 |
| | HRV24: AAGGTGTAAAATCTGTTGGAAG | 159 |
| | HRV25: AGATGAAGTGTCTGGTTGTG | 160 |
| | HRV2G: AATCTACATGCTGATGTGTCC | 161 |
| | HRV27: GGCAAGTGTGTAAAATCTGC | 162 |
| | HRV28: GAAGAAGTGTCAGGGTGTG | 163 |
| | HRV29: TGAGGTGTCTGGCTGG | 164 |
| | HRV30: GGTGTAAAACCTATTGGAAGATG | 165 |
| | HRV31: TCTATTTGATGAGGTGTCTGG | 166 |
| | HRV32: GAAGTGTCTGGATGAGAAGG | 167 |
| | HRV33: CTATTAGAGGAAGTATCTGGTCG | 168 |
| | HRV34: ATCTATTAGAAGAGGTGTCTGG | 169 |
| | HRV35: CCTACATACTGAAGTGTCTGG | 179 |
| | HRV36: AGATGAAGTGTCTGGTTGTG | 171 |
| | HRV37: ATCTACATACAGAAGTGTCTGG | 172 |
| | HRV38: ATGTGTCTGGTTGTGAAGG | 173 |
| | HRV39: CCATACCTTACTCTCTAAGGTG | 174 |
| | HRV40: GATGATGTATCAGGCTGGC | 175 |
| | HRV41: GAATCTATTGGAAGAAGTATCAGG | 176 |
| | HRV42: TACATGCTGAAGTGTCTGG | 177 |
| | HRV43: CCATGACCTACTCTCCAAAG | 178 |
| | HRV44: TATTTGACGAGGTGTCTGG | 179 |
| | HRV45: CATTCCTTACTTTCCAGTGTG | 180 |
| | HRV46: AGGTATAGAATCGGTTGGATG | 181 |
| | HRV47: GAATCTATTTGATGAAGTGTCTGG | 182 |
| | HRV48: GTAGAATCTGCATACAGATGTG | 183 |
| | HRV49: CGGACGAGAAGGTTTGTC | 184 |
| | HRV50: GTGTCAGGACGTGATGG | 185 |
| | HRV51: CACAATTTACTGTCCAAGGTG | 186 |
| | HRV52: CCATGTCTTGCTGTCCAATG | 187 |
| | HRV53: TCTTGAGCAGATAAATACTCTGG | 188 |
| | HRV54: GATGTGTCAGGTTGTGTAGG | 189 |
| | HRV55: GATGAAGTATCTGGTCTAGATGG | 190 |
| | HRV56: GTTGGAGGAAGTGTCTGG | 191 |
| | HRV57: AGGAAGTATCTGGTCTAGATGG | 192 |
| | HRV58: AAGGTGTAGAATCTATTGGAGG | 193 |
| | HRV59: GATGATGTGTCTGGTTGTGTAG | 194 |
| | HRV60: TAGATGATGTGTCAGGTTGTG | 195 |
| | HRV61: GATGTGTCAGGCTTAGAAGG | 196 |
| | HRV62: GAAGTGTCTGGTTGTGTAGG | 197 |
| | HRV63: GTGTAGAAGCGATTGGAGG | 198 |
| | HRV64: GTGTAGAATCTGTTTGAGGATG | 199 |
| | HRV65: TCGGCATCAGATAAGTATTGTG | 200 |
| | HRV66: GCAGTAGCATCTGCACC | 201 |
| | HRV67: AGTGTAGAATCTGTTAGAAGAGG | 202 |
| | HRV68: TGAAACATCTGGATGAGTTGG | 203 |
| | HRVG9: ACCTGCATACTGACGTATC | 204 |
| | HRV70: CTACACACTGATGTGTCAGG | 205 |
| | HRV71: TCTATTTGAAGAGGTGTCTGG | 206 |
| | HRV72: CCCATTTTGCTGTCTAATGTG | 207 |
| | HRV73: CTATTGGAGGAAGTATCAGGTC | 208 |
| | HRV74: AGCAGTTGCATCTTCTGG | 209 |
| | HRV75: CGGTTGTGATGGCTTGTC | 210 |
| | HRV76: TCTATTAGAGGAGGTGTCTGG | 211 |
| | HRV77: ACGTGTCTGGTTGAGTG | 212 |
| | HRV78: GCTCTCCAGAGTATAGAACC | 213 |
| | HRV79: CTACACACAGATGTGTCTGG | 214 |
| | HRV80: GTACTTTCTAGTGTGTACAACC | 215 |
| | HRV81: TATTTGATGATGTATCTGGCTGG | 216 |
| | HRV82: TCTATTGGATGATGTGTCAGG | 217 |
| | HRV83: CTGCACACAGATGTATCTGG | 218 |
| | HRV84: CCAAGTTTTACTAGCCAAAGTG | 219 |
| | HRV85: CTATTTGATGATGTGTCAGGTTG | 220 |
| | HRV86: TACTATCTAAGGTGTAAAATCTACACAC | 221 |
| | HRV88: GAAGTATCTGGTTGTGTTGG | 222 |
| | HRV89: GTCCAAGAACGACTGTCC | 223 |
| | HRV90: CTATTAGATGAGGTGTCTGGAC | 224 |
| | HRV91: TTGCTGTCTAAAGTATAAAATCTACATAC | 225 |
| | HRV92: CTGCACACTGAGGTGTC | 226 |
| | HRV93: CTACATGCAGATGTGTCTGG | 227 |
| | HRV94: GTTCGAGGAAGTGTCGG | 228 |
| | HRV95: ATCTATTGGTTGAGGTATCAGG | 229 |
| | HRV96: GGAAGATGTGTCAGGTCTG | 230 |
| | HRV97: TACAAGCAGATGTATCAGGTC | 231 |
| | HRV98: TGAAGATGTATCTGGTTGCG | 232 |
| | HRV99: TCTACAAGCTGAAGTATCTGG | 233 |
| | HRV100: TGTTGGAAGATGTATCAGGTG | 234 |
| | Hanks: TGAGGAAGTGTCTGGTCTAG | 235 |

### Example 3

### Selection of Specific MultiCode Primer Sets

Each MultiCode primer set used in the following example has two PCR primers and one TSE primer. Multiple candidate primer sets were generated for each of the 18 detection targets described above. Stretches (>60 bases) of conserved sequences in each target genomic region were identified by aligning all the available sequences with program ClustalX. Secondly, candidate primer sets were selected within these conserved sequences with computer software (Visual Oligonucleotide Modeling Platform [OMP], DNA Software, Inc.), according to the following criteria: appropriate melting temperature, minimal secondary structure formation, minimal interactions with the other primers in multiplex setting and no interaction with human sequences.

The performance of each candidate primer set was evaluated in a MultiCode PLx assay (e.g., as described below in Example 5) by preparing samples containing 20 copies of target cDNA, a target concentration near the lower limit of assay detection. The primer set that gave the maximal fluorescent signal for each target was selected. Each of these 18 selected primer sets (Table 4) generated signal (2000 to 10,000 MFI) that was markedly higher than the background signal (300 MFI and lower) of the negative control (60,000 copies of human DNA per reaction).

### Example 4

### Development of the pan-HRV Primer Set

HRV group has 101 known serotypes that account for >60% of all proposed target viruses. To design a pan-HRV MultiCode primer set, we collected and analyzed all available sequences of HRV 5′ NCR (146 published and 101 new sequences) that covered all 101 known serotypes. The analysis results revealed that the 5′ NCR had 3 stretches (A, B and C) of almost completely conserved sequences, corresponding to nucleotide# 352-368 (A), 442-462 (B) and 535-554 (C) of HRV16. Within these 3 stretches of sequences, 12 PCR forward (A), 9 PCR reverse (C) and 5 TSE (B) candidate primers were designed using program Visual OMP. The primer set with the best signal/noise ratio (Table 4) was then selected from these candidate primers by testing different combinations of PCR and TSE primers against the DNA clones of the 5′ NCR of HRV1A, 2, 17 and 59 in a MultiCode PLx assay as described below in Example 5. These 4 serotypes were used because the collective sequences of their primer sites had identity with 99 of the 101 serotypes. Typical performance for this primer set was ascertained against a representative target, HRV1A. With 10 copies of target per reaction, this HRV primer set produced a signal of about 2000 MFI, which was 10-fold higher than the background. The signal strength of the assay increased with target concentration and then reached a plateau at 200 copies of target per reaction.

To determine whether this primer set could sensitively detect all HRV, cDNA clones of the 5′ NCR of all 101 serotypes were tested. The results showed that this primer set detected 99 serotypes at 20 copies of target per reaction with a typical signal of about 2000 MFI. However, higher target concentrations were needed for HRV33 (100 copies) and HRV78 (40 copies). Consistent with these results, a single-base mutation was found in the PCR forward primer site of these two viruses (data not shown).

TABLE 4
Sequences of RMA primers for 18 detection targets

| | | | | |
|---|---|---|---|---|
| | | SEQ | | SEQ |
| Viral | | ID | TSE primers | ID |
| targets | PCR Primers^{a} | NO: | target-specific extension) | NO: |

| | | | | |
|---|---|---|---|---|
| HRV | F: AGCCTGCGTGGC | 1 | CGGCYCCTGAATGYGGCTAA | 3 |
| 5′NCR | R: CGGACACCCAAAGTAGT | 2 | | |
| EnV | F: GGCTGCGYTGGCG | 4 | ACAWGGTGYGAAGAGTYTATTGA | 6 |
| 5′NCR | R: CGGACACCCAAAGTAGT | 5 | GCTA | |
| CoV | F: TGATCAAATTGCTAGTCTTG | 7 | AGGATGCCACTAAACCTCAGC9 | |
| OC43N | R: CTTATTCAAAATTTTCTGTCTG | 8 | | |
| CoV | F: ATTTCATGCTTTTGTTCTT | 10 | ACTCTTGGCAGAAGTTTGAGAAG | 12 |
| 229E N | R: ATAAAAAGTCAGCGAAAAC | 11 | A | |
| CoV | F: TGGCTTTAAAGAACTTAGG | 13 | CAGTCGAAGTCACCTAGTTCTTC | 15 |
| NL63N | R: AAAGAGGCTTATTAGGTTTC | 14 | TG | |
| RSVA F | F: CACCCGTTAGAAAATGT | 16 | GTTTTGCCATAGCATGACACAAT | 18 |
| | R: TTCAAAAACAGATGTAAGCA | 17 | G | |
| RSVB F | F: ATTGCATTTGGTTTCTTTTA | 19 | CTATTGTTATGACACTGGTATAC | 21 |
| | R: GTTTTACCAATCGACATGT | 20 | CAACCTGTTC | |
| MPV L | F: ATGACBACAATGATATGTGC | 22 | AGACATGCACCACCAGAAACAAA | 24 |
| | R: CTGGTTTACTKACATCTATTGA | 23 | | |
| PIV1 HN | F:TGAGTGATTAAGTTTGATGA | 25 | TGCATCACCAATTGATAATGAAG | 27 |
| | R: ATTATTACCYGGACCAAG | 26 | GT | |
| PIV2 HN | F: GGGTTGATTGTGGCCCA | 28 | TGCCCTGTTGTGTTTGGAAGAGA | 30 |
| | R: TGAGACTTGCTTTCTATTATTA | 29 | TATGACT | |
| | TAATGATAC | | | |
| PIV3 HN | F: ATGCTTATACCTCRAATCTA | 31 | ACTCGAGGTTGYCAGGATATAGG | 33 |
| | R: TRGGATTTAAGTCAGGTACC | 32 | AA | |
| PIV4a HN | F: GGGCGATTTCAATTTTT | 34 | CCTCTCTGATAATAAAATATGTT | 36 |
| | R: GCAGAGGGTCGATTATATA | 35 | GTTCTCAATG | |
| PIV4b HN | F: TGTGCAGGTGCTTTC | 37 | CAATGATCTTTTATTTTCGCAAT | 39 |
| | R: CCCATAAGGCAAGAAG | 38 | TATGTTTGTTT | |
| InFVA M | F: ATTGCCTGCACCATYT | 40 | ACTTGATCCAGCCATTTGCTCCA | 42 |
| | R: GTTYTGGCCAGCACT | 41 | | |
| InfVB M | F: AAAGAAGATTCATCACAGAG | 43 | CAGGAATGGGAACAACAGCAACA | 45 |
| | R: TGCTATTTCAAATGCTTCA | 44 | AA | |
| AdVB | F: MAGYACTCTGTTGTCSCC | 46 | GTCAACGGGCAYRAAGCGCA | 48 |
| hexon | R: GGGTCTGGTGCAGTT | 47 | | |
| AdVC | F: CTGAAGTACGTCTCGG | 49 | GGCGTCCTGGCCCGAG | 51 |
| hexon | R: GCTACCCCTTCGATG | 50 | | |
| AdVE | F: CATTGGCATAGAGGAAGT | 52 | AAGGATTGCCTACATGGATTTCA | 54 |
| hexon | R: AGATGCAGGTTCTGAA | 53 | TTAGC | |

^{a} F: Forward PCR primer and R: Reverse
Y = C + T; B = C + G + T; K = G + T; W = A + T; M = A + C; R = A + G
All reverse primers include an iso-C: e.g., 5′-iC-AGATGCAGGTTCTGAA

### Example 5

### Detection of Target Viruses by Panels A and B using the Respiratory MultiCode-PLx Assay Method

Separate panel assays were used for HRV and EnV detection due to sequence homology at the primer sites and some cross-reactivity. Therefore, the 18 primer sets were divided into 2 detection panels, called A and B. Panel A included 11 primer sets for HRV, RSVA, RSVB, MPV, PIV1, PIV2, PIV3, PIV4a, PIV4b, InfVA and InfVB. Panel B included 7 primer sets for EnV, CoVOC43, CoV229E, CoVNL63, AdVB, AdVC and AdVE.

The performance of Panels A and B primer sets was evaluated in MultiCode-PLx assay (termed Respiratory MultiCode-PLx Assay or “RMA”) against the DNA clones of the different detection targets and human genomic DNA (negative control) at 20 and 60,000 copies per reaction respectively.

The RMA assay was performed in 96-well PCR plates (BioRad MLL9601) in triplicate. Each assay included the following steps (all of which occur in the same well) with reagent additions followed by mixing, sealing with Microseal B film (BioRad MSB1001) and incubation: amplification of viral cDNAs by PCR, labeling of the PCR products with virus-specific tags and site-specific biotins by target-specific extension (TSE) of tagged primers, capture of the tagged TSE products (target oligonucleotides) by the microspheres through the hybridization of each tagging sequence to its precise complementary oligonucleotide conjugated to the surface of a color-addressed microsphere, binding of fluorescent streptavidin-phycoerythrin (SAPE) to the biotins of TSE products, and reading of fluorescent signals on each microsphere using the Luminex LabMap 100 instrument.

The PCR step was carried out in 8 μl reaction mix containing 2 μl cDNA, 1 μl MC-PCR buffer (EraGen PN1235), 0.16 μl of Taq polymerase (BD Bioscience 639209) and between 100 and 300 nM PCR forward and 5′-isoC modified reverse primers. The primer concentrations for each cDNA target were determined empirically; however, the concentration of forward and reverse primer were the same for any particular cDNA target. Conditions for PCR reactions were as follows: 5 min at 95° C. and 28 cycles of (5 sec at 95° C., 10 sec at 55° C., 30 sec at 72° C.). In some embodiments, the PCR reaction (e.g., the MultiCode primers) is exhausted before continuing with the TSE reaction.

Immediately after the PCR, 2 μl of TSE mix containing 1 μl of MC-TSE buffer (EraGen PN1308) and 75 nM TSE primers (EraGen) was added to the PCR product. The TSE reaction was carried out in the following conditions: 30 sec at 95° C. and 10 cycles of (5 sec at 95° C., 2 min at 65° C.).

After the TSE reaction, 40 μl of microspheres/hybridization solution (EraGen PN1402/1237) was added to the TSE products. The resulting mixture was incubated at room temperature for 10 minutes in the dark to allow hybridization of TSE products to the tag specific microspheres. Then 40 μl of sheath fluid (Luminex 40-50000) containing 2 μg of SAPE (Prozyme PJ31S) was added. Next, the fluorescent signal associated with each microsphere was measured in a Luminex machine (96 well-plate flow cytometer). The signal is expressed as MFI (median fluorescence intensity). Samples with an average signal >6 standard deviations of average negative control signals (typically 400 to 500 MFI) were regarded as positive.

Each primer set of Panels A and B provided a strong target-specific signal (signal/noise ratio of 14-240 with an average of 46) and had no nonspecific reaction with human sequences or the other viral targets.

For Panel A primer sets, the target-specific signals ranged from 2500 to 7900 median fluorescence intensity (“MFI”) with an average of 4200 MFI and low background signals (60-270 MFI with an average of 180 MFI) as measured against 60,000 copies of human DNA. The PIV4b primer set gave a slightly higher background signal (560 MFI), but it also generated the strongest target-specific signal (7900 MFI).

For Panel B primer sets, the target-specific signals ranged from 3600 to 10,300 MFI with an average of 5400 MFI and the background signals were between 20 to 180 MFI with an average of 110 MFI.

### Example 6

### Preparation of cDNA from Clinical Specimens

A sample preparation protocol was developed which generates sufficient cDNA to allow detection in samples containing only a hundred copies of viral target sequences. (Compare to previous methods, e.g., Gern, J. E., et al. 2002. Pediatr. Allergy and Immunol., 13:386-393). Some of the changes yield improvements in the recovery of viral RNA by optimizing the RNA extraction conditions and selecting a reverse transcriptase enzyme (AMV-reverse transcriptase, Promega M510F), with greater cDNA synthesis efficiency at low template concentrations.

Generally, all steps were carried out in Eppendorf DNA LoBind tubes (Eppendorf 022431021). To 200 μl of specimens (e.g., nasal lavage fluid or swab), 150 μl PBS (phosphate buffered saline), 20 μl of glycogen (Ambion 9510), 15 μg of glucoblue (Ambion 9515), 50 ng of human genomic DNA (BD Bioscience 6550-1) and 750 μl of Trizol LS (Invitrogen 10296) were added. The resulting mixture was vortexed vigorously for 10 minutes, supplied with 230 μl of chloroform, vortexed vigorously for 5 minutes and then microfuged for 5 minutes. The resulting aqueous phase (˜700 μl) was transferred to a new tube containing 600 μl isopropanol to precipitate RNA. The RNA precipitant was pelleted by centrifugation for 10 minutes. The RNA pellet was washed once with 700 μl of 75% ethanol, air-dried and dissolved in 20 μl water.

To make cDNA, 16 μl of RNA solution was added to 24 μl of reaction solution containing 6 units of AMV-reverse transcriptase (Promega M510F), 8 μl 5×AMV-RT buffer (Promega M515A), 0.5 μg random primers (Promega C1181), 20 units of RNAsin (Promega N2615), 8 μl 5 mM dNTPs (Promega U1330) and then incubated at 25° C. for 5 minutes, 42° C. for 10 minutes, 50° C. for 20 minutes, and 85° C. for 5 minutes.

The new protocol produced sufficient cDNA from a sample containing only 100 virions to generate a strong signal using the RMA methods described herein. Since an infectious unit of HRV16 typically contains 200-400 virions, the present detection assay was more sensitive than traditional viral culture assays that could detect no less than 1 infectious unit.

### Example 7

### Identification of Target Viruses in Clinical Specimens by RMA

The accuracy of both RMA Panels A and B to detect respiratory viruses in human specimens was evaluated against 101 clinical samples (throat swabs or nasal wash) that previously tested positive by traditional culture or immunofluorescence staining methods (e.g., Gern, J. E., et al. 2002, Pediatr Allergy Immunol. 13(6):386-93) for HRV (20 specimens; n=20), RSV (n=21), InfVA (n=10), InfVB (n=10), PIV1 (n=10), PIV3 (n=10), and AdV (n=20).

After RNA extraction and cDNA synthesis, duplicate samples were then tested by both RMA Panel A and B assays. Samples with an average signal >6 standard deviations of average negative control signals (typically 400 to 500 MFI) were regarded as positive. Positive signals ranged from 1000 to 10,000 MFI and background signals from 0 to 300 MFI (Table 4). The RMA detected HRV and AdV in all positive samples, RSV in 95% of RSV samples, and InfVA, InfVB, PIV1 and PIV3 in 90% of samples. In AdV samples, all 3 groups, B, C and E, were detected and in RSV samples, both A and B were found. In addition, RMA also detected HRV (n=3), InfVA (n=3), and PIV3 (n=3) in 9 samples that were tested negative for these viruses by Wisconsin State Laboratory of Hygiene (“WSLH”), and detected MPV (n=1), OC43 (n=2) and EnV (n=1) that were not tested by WSHL. Therefore, compared to traditional methods, the respective sensitivity of RMA to detect HRV, RSV, InfVA, InfVB, PIV1, PIV3 and AdV were 100%, 95%, 90%, 90%, 90%, 90%, and 100% and the respective specificity was 96%, 100%, 97%, 100%, 100%, 97%, and 100%. The overall sensitivity and specificity of RMA were 94% and 99% respectively. (Table 5).

TABLE 5
Detection of respiratory viruses by RMA in clinical specimens
characterized by traditional methods

| | No. of | | Additional viruses |
|---|---|---|---|
| | WI State | RMA | Detected by |
| Target viruses | Lab samples | Positive^{a} | RMA |
| HRV | 20 | 20 | |
| RSV | 21 | 20^{b} | HRV(1), InfVA(2) |
| | | | PIV3(2), OC43(1) |
| InfVA | 10 | 9 | |
| InfVB | 10 | 9 | |
| PIV1 | 10 | 9 | |
| PIV3 | 10 | 9 | EnV(1) |
| AdV | 20 | 20^{c} | HRV(2), InfVA(1) |
| | | | PIV3(1), MPV(1), OC43(1) |

^{a}Samples were called positive when the virus signal was >6-fold of the standard deviation of negative control (500 MFI in this experiment).
^{b}1 sample had both A and B; 11 samples were called A, 8 samples were called B.
^{c}3 samples had both B and E; 6 samples were called B, 11 samples were called C.

### Example 8

### Detection of Respiratory Viruses in Nasal Lavage Specimens by RMA

The sensitivity of the RMA to detect respiratory viruses in clinical specimens was further assessed by testing 103 additional samples of nasal secretions from 5 year-old children with asthma and respiratory symptoms. These specimens were collected using a “nose blowing” technique. For comparison, a second aliquot of each sample was sent to WSLH for traditional culture and immunofluorescence staining tests (e.g., Gern, J. E., et al. 2002, supra).

By traditional methods, viruses were found in 24 of the 103 samples (Table 6). In contrast, RMA detected respiratory virus in 74 samples (71.8% of the total), including 70 samples with 1 virus and 4 samples with 2 viruses. RMA had improved rates of detection for RSV, PIV3 and PIV4 and especially HRV and EnV. RMA detected HRV and EnV in 37 and 4 samples respectively while traditional methods detected these viruses in only 6 and 1 samples, respectively. Detection for InfV A, InfV B, and PIV1 were approximately equal for either assay.

TABLE 6
Detection of respiratory viruses in 103 nasal lavage specimens from
5 year-old children with asthma and respiratory symptoms

No. of virus detected

| | | Traditional |
|---|---|---|
| Viruses | RMA^{a} | methods^{b} |
| HRV | 37 | 6 |
| RSV | 13 | 8 |
| | (7 A and 6 B) | |
| InfV A | 2 | 2 |
| InfV B | 1 | 1 |
| MPV | 5 | Not tested |
| PIV1 | 3 | 3 |
| PIV3 | 3 | 2 |
| PIV4 | 2^{c} | 1 |
| AdV | 4 | Not tested |
| | (1 B and 3 C) | |
| CoV OC43 | 2 | Not tested |
| CoV NL63 | 2 | Not tested |
| EnV | 5 | 1 |
| Total | 79 | 24 |

^{a}Total of 75 samples tested positive for a virus. 71 samples had 1 virus and 4 samples had 2 viruses.
^{b}Total of 24 samples tested positive for virus by culture and immunofluorescence staining. Each sample has 1 virus.
^{c}Both are PIV4b.

To verify that the detection of HRV by RMA in 34 culture negative samples was not false-positive, an third assay was performed that directly identified the HRVs in these specimens by cloning and sequencing their 5′ NCRs. Briefly, the 5′ noncoding region (NCR) of HRV was amplified from the cDNA used for the RMA by semi-nested PCR (e.g., Gern, J. E., et al. 2002, supra) with 3 universal PCR primers (corresponding to nucleotides 163-181, 443-462 and 536-552 of HRV 16). These 3 universal primers were designed according to the conserved sequences identified in the database of 247 HRV 5NCR sequences, described above. PCR products of each sample were analyzed in a 1.5% agarose gel for the presence of the predicted 300-base PCR fragment (corresponding to nucleotides 163-462 of HRV16). The 300-base PCR fragment of each positive sample was purified with 1.2% low melting point agarose gel, inserted into plasmid vector pMJ3 and then transformed into E. coli. Plasmids with the correct inserts were isolated, amplified and purified. Each insert was completely sequenced.

In sum, 29 of the 31 HRV samples and all 3 EnV samples produced the predicted 300-base PCR fragment. Each of the 32 PCR fragments was cloned into a plasmid vector and then sequenced. These sequences were compared to the database of the HRV 5′ NCR sequences (described above) and the Genbank sequence database with computer software (Clustal X and BLAST). The results verified that all 29 HRV sequences, and an additional 6 samples that were both culture and RMA positive, had HRV, and all 3 EnV samples had EnV. Alignment of the 35 HRV sequences with the HRV 5′NCR sequence database revealed 24 distinct HRV strains (pairwise nucleotide variations between 9-59%): 16 strains were detected once, 5 strains twice, and 3 strains were found in three samples each. None of the sequences identified more than once were from sequential samples, suggesting none of the repeat isolates was due to cross-contamination.

### Example 9

### Testing an Additional 689 Clinical Samples with RMA

To further validate the respiratory viral detection methods, a total of 689 samples, including throat and/or nasopharyngeal swabs submitted to the WSLH by rapid influenza test sites and sentinel clinician virus surveillance sites throughout Wisconsin were tested. Samples were collected during the influenza season (Dec. 1, 2005 to Apr. 1, 2006).

As noted above, due to some sequence homology at the primers sites and some cross-reactivity, separate panel assays were developed for HRV and Env. In this example, the Env primer set was excluded and the following 17 viral groups were detected simultaneously: influenza viruses A and B, RSV A and B, adenovirus subgroup B (types 3, 7, 11, 14, 16, 21, 34, 35), subgroup C (types 1, 2, 5, 6) and subgroup E (type 4), parainfluenza viruses (PIV) 1, 2, 3, 4a and 4b, rhinovirus, coronavirus 229E, OC43, and NL63, and human metapneumovirus (hMPV).

Establishing limits of detection for primer set combinations. Clones that contain the target genomic regions (e.g., as described above in section II.B) of the following 31 viruses were used to determine assay sensitivity: Adenovirus subgroup C (serotypes 1 and 5), Adenovirus subgroup B (serotypes 3, 7, 11, 21, and 34), Adenovirus subgroup E (serotype 4), rhinovirus (serotypes 1a, 2, 13, 14, 17, 59, 86, and 91), CoV NL63, CoV 229E, CoV OC43, InfB, hMPV, RSVA, RSVB, PIV1, PIV2, PIV3, PIV4a, PIV4b, InfA (H3N2), and InfA (H5N1). Purified plasmid DNA was quantified by Quant-iT™ PicoGreen® dsDNA Assay Kit (Invitrogen). All plasmids with the exception of those containing PIV4a and PIV4b sequences were linearized by restriction enzyme digest of 100 ng/μl solution of each target. The templates were then diluted to the concentrations used in the LOD experiment (0.25, 2.5, 25, and 2500 template copies per reaction) and target numbers were verified by OD 260. Reactions were performed in duplicate. Results were considered positive when MFI output was at least 6 standard deviations over the average background. The lowest dilution that gave a positive result for both replicates was considered as the limit of detection (“LOD”). For all cut plasmid targets tested, the LOD was determined to be 2.5 copies.

Due to sequence variability, multiple duplicate tests were performed using various subtypes for Adenovirus and HRV. Since all targets could be detected within each single multiplexed reaction (all primers were added to each reaction) and only one target was added to any given reaction, analytical specificity for the other targets could also be determined.

Overall, analytic specificity for the assay was determined to be 100%. That is, no false positive results occurred during all 62 reactions. In addition, all reactions detected the correct target at all input target copy numbers above the LOD limit which provided a analytical sensitivity of 100%. Interference testing was not conducted.

Respiratory Sample Preparation. The 689 WSLH samples were vortexed in 2 mL of viral transport media (M4) for 1 minute. The samples were divided equally into two parts and used in virus culture/DFA staining or molecular testing. For molecular testing, prior to sample extraction the inoculated media was centrifuged for 10 minutes at 17,000×g for 20 minutes at room temperature and the cell pellet was resuspended in approximately 200 μl of supernatant. The nucleic acid extraction was performed with a MagNaPure® LC (Roche Diagnostics) instrument using the total nucleic acid kit (Cat. No. 3 038 505). The sample and elution volumes were 200 and 50 μl, respectively.

CDC Influenza A and B Real-Time PCR Testing. 5.0 μL of extracted nucleic acid was tested by real-time PCR with the ABI 7500 Fast (Applera) using Center for Disease Control (“CDC”) protocols for influenza A and B viruses. These protocols were validated at the WSLH in 2005 by comparison to virus culture.

Virus culture and direct immunofluorescence. Specimens that tested negative for influenza A and B viruses by real-time PCR were inoculated into MDCK, A549, PRMK, and WI-38 cells using routine methods. Specimens were also tested by direct immunofluorescence for RSV.

RMA Testing. Samples to be tested were batched. Batch size was between 50 and 80 samples. Over the course of 4 months, two operators performed 9 batched runs. Each batched run included positive and negative control. The signal generated from the negative control was used to determine cross-contamination and the signal generated from the internal positive control (“IPC”) was used to determine assay reproducibility.

The sequence of the IPC primers used is: 5′-iC-ATTGGACGATATCGTTCTC (reverse, SEQ ID NO. 55), and 5′-AACGGATAATACTAAAGGCC (forward, SEQ ID NO: 56). The sequence of the TSE probe used is: AYCGYCYA-C3-CCCAATCCACGGACACAGG (SEQ ID NO: 57). IPC DNA was added to the amplification mix at a level of 1200 copies per reaction and primer sets specific to the IPC yield a positive IPC channel signal.

Positive target controls using a subset of the cloned target sequences (described above) were also implemented into each batch reaction set. To eight reactions of every batch set, all 17 targets with the exception of PIV4b were tested (2 targets per reaction).

Finally, cross-over contamination was analyzed using negative control reaction spaced out throughout the plate. Each batched run conducted included negative controls in order to monitor possible cross contamination. The negative controls were performed using extraction elution buffer in 1 out of every 16 samples per batch.

Reverse transcription. 6 μL of extracted nucleic acid (prepared as described above and batched) was added to 6 μL of reverse transcription solution which included: 15 μM random hexamers and 1.5 units AMV RT. The reactions were heated to 42° C. for 10 minutes, 50° C. for 20 minutes, 85° C. for 5 minutes then held at 4° C. until amplification. Before the reactions were heated, plates were sealed with Microseal B film (Biorad MSB 1001) to prevent evaporation.

RMA Reaction. All batched reactions were performed in 96-well PCR plates (BioRad MLL9601). The reactions included the same steps as noted above (amplification of viral cDNAs by PCR, labeling of the PCR products with virus-specific tags and site-specific biotins by target-specific extension (TSE) of tagged primers, capture of the tagged TSE products (target oligonucleotides) by the microspheres through the hybridization of each tagging sequence to its precise complementary oligonucleotide conjugated to the surface of a color-addressed microsphere, binding of fluorescent streptavidin-phycoerythrin (SAPE) to the biotins of TSE products). In this example, fluorescent signals were read on each microsphere using the Luminex LabMap 100 instrument.

Reactions were initiated by combining 5 μL of the reverse transcription reaction prepared above with 5 μL of PCR master mix that includes 0.2 μL of Titanium Taq polymerase (BD Bioscience 639209) along with all PCR primer pairs. The PCR step was carried using the following conditions: 2 min at 95° C. and 30 cycles of (10 sec at 95° C., 30 sec at 55° C., 30 sec at 72° C.), then held at 4° C.

Following the PCR reaction, 5 μL of a TSE master mix that includes the TSE primers along with 6 uM Biotin-diGTP was added to the PCR product. The TSE reaction was carried out using the following conditions: 30 sec at 95° C. and 10 cycles of (5 sec at 95° C., 2 min at 65° C.), 65° C. for 5 min then held at 4° C.

After the TSE reaction, 35 μL of microspheres/hybridization solution (EraGen PN9550/9570) was added to the TSE products. The resulting mixture was incubated at room temperature for 10 minutes in the dark to allow hybridization of TSE products to the tag specific microspheres. Then 35 μL of sheath fluid (Luminex 40-50000) containing 2 μg of SAPE (Prozyme PJ31S) was added. Next, the fluorescent signal associated with each microsphere was measured in a Luminex machine (96 well-plate flow cytometer). The signal is expressed as MFI (median fluorescence intensity). Samples with an average signal >6 standard deviations of average negative control signals (typically 400 to 500 MFI) were regarded as positive.

RMA Data Analysis. Template set-up within the Bio-Rad BioPlex 3.0/4.0 software was required and importation to the analysis software was achieved by importing the data export file into the EraGen MultiCode PLx analysis software. Data files were parsed and the resulting raw MFI values were organized by target and sample. Following data acquisition from all clinical samples tested, default cut-off windows for each target were empirically determined and set in a blinded fashion. Once determinations were made, reports were generated for offline analysis.

The signals generated in the IPC channel from 806 separate reactions averaged 7746 mean fluorescent units (MFI) with a standard deviation (SD) of 628 MFI. Of the 806 reactions, 8 reactions (˜1%) did not produce an IPC signal above 2500 MFI. Of these 8 failed reactions, the average IPC MFI was 1125 with a SD of 628.

Positive target run controls using a subset of the cloned target sequences from above were implemented into each batch reaction set. To eight reactions of every batch set, all 17 targets with the exception of PIV4b were tested (2 targets per reaction). For the 70 reactions run and a possible 140 positive signals, one reaction reported a failed IPC and one reaction failed to detect PIV1. All other reactions reported the correct positives and negatives. Positive call cut-offs were set by the following calculation: average negative signal plus six times the negative SD.

Analysis of RMA data from the initial surveillance network samples indicated that out of the 689 samples, 446 were positive for one or more targets. The average positive signals were at least 17-fold above the background noise for any given target. Expectedly, the targets with the highest percentage of sample positive calls were influenza A and influenza B. For those two targets, the average signal to noise value was 47-fold and 25-fold respectively, with the average target specific signals for those targets were 5680 and 3450 MFI respectively.

Contamination potential and/or amplicon carry-over was monitored by observing signal generation in blank reactions. Viral transport media (VTM) was added to the blank reactions prior to extraction. The blanks were segregated throughout the extraction plate (2 per plate) and throughout the RMA batched reaction plates (7-8 per plate). A total of 50 blank reactions were performed of which 49 produced negative results and 1 produced a negative result but the IPC failed.

For direct comparison to the WSLH testing algorithm, a “true positive” was defined as a specimen that was positive by either virus culture (including RSV DFA) or influenza PCR. Of 689 specimens tested, 73% (n=503) were positive by virus culture or PCR compared to 65% (n=446) positive by the RMA. Of the 446 RMA positive samples, nine (2%) were identified as coronavirus NL63, 5 (1%) were identified as human metapneumovirus (both of which current WSLH culture methods do not detect) and 16 were identified as dual infections. The sensitivity of the RMA ranged from 52% (Flu B) to 100% (Adeno and Rhinoviruses). The RMA was able to detect 97% of influenza A samples that tested positive by CDC RT-PCR; however it failed to detect one influenza A/H1 swine strain. The RMA also failed to detect 48% of the influenza B CDC RT-PCR positive samples. The CDC RT-PCR cycle thresholds ranged from 15 to 40, indicating a wide viral target input copy range. Additionally, sixteen samples (4%) were found to be positive for more than one virus.

Testing CoV and hMPV Positive Samples via Real-time PCR. Samples positive by PLx-RVP and not detectable using the WSLH standard testing algorithm were further tested by the Washington University School of Medicine, St. Louis, Mo. with real-time PCR assays. Reverse transcriptase real-time PCR(RT-PCR) assays for CoV and HMPV were carried out in 50 μl reaction volumes in ABI optical quality 96-well plates. Each reaction contained 1× Qiagen Quantitect Probe RT-PCR Kit reaction mixture (Qiagen Valencia, Calif. Cat No. 204443). Thermal cycling was performed on an ABI 7300 RT-PCR instrument using “absolute quantitation” software. The conditions were as follows; for coronaviruses: 20 min at 50° C., 15 min at 95° C. followed by 45 cycles of (15 sec at 94° C., 60 sec at 60° C.); for HMPV: 30 min at 50° C., 15 min at 95° C. followed by 45 cycles of (15 sec at 94° C., 60 sec at 55° C.). Threshold crossing cycles were computed by the ABI 7300 RT-PCR System Sequence Detection Software version 1.2.1. RT-PCR for HMPV was performed using primers and MGB modifications of the probes as described in Mahony et al., (2004) J Clin Microbiol., 42:1471-6. Briefly, probes were labeled with FAM at their 5′ end and had the minor groove binding (MGB) peptide at their 3′ end. For the CoV assay, primers and probes that amplify segments of the N gene of human CoV OC43, 229E, and NL63/New Haven were employed. Coronavirus strains were obtained from ATCC (OC43 and 229E) or from Dr. Lia van der Hoek (NL63). For each CoV in vitro RNA transcripts from cloned segments that contained the assay target for each strain were created and quantified. The analytical sensitivity (LOD is defined as the RNA level at which 95% of replicates were positive) of each assay was determined to be 10 copies/reaction (manuscript in preparation). All assays were optimized for primer and probe concentration and annealing temperature (manuscript in preparation).

The PLx-RVP positive samples for targets which current WSLH culture methods do not detect (9 CoV NL63 and 5 human metapneumovirus positive samples) were tested by the above single target real time RT-PCR assays. Of all tests completed, real time PCR data showed all but one (HMPV real-time RT-PCR negative sample) to be concordant with the PLx-RVP results.

### Example 10

### Test of Primer Sets on 155 WSLH Samples

Given that the RMA failed to detect 48% of influenza B samples that tested positive via the CDC RT-PCR assay, a number of influenza B isolates were sequenced. It was found that influenza strains present in the missed samples were not in GenBank when the primers and probes were designed. Further, new influenza A (e.g., the Swine and Avian strains), RSV and PIV2 strains were also identified in these databases since the original primer design. Accordingly, primers and probes used in the following example are as shown in Tables 7 and 8, respectively.

TABLE 7
Sequence of RMA Amplification Primers

| Viral | | SEQ ID |
|---|---|---|
| targets | Amplification Primers | NO: |
| InfVB | F: CAATTCCTTCCCCATTCTTTTGAC | 58 |
| | R: XCTATGAACACAGCAAAAACAATGA | 59 |
| InfVA1 | F :CCGAGGTCGAAACGT | 60 |
| | F: CAGAGGTCGAAACGT | 61 |
| | R: XTGGGCACGGTGAG | 62 |
| InfVA2 | R: XGCAACAACCAATCCATTAATAA | 63 |
| | F: CCAGCCATTTGITCCATA | 64 |
| RSVA | R: XTTCAAAAACAGATGTAAGCA | 17 |
| | F: CACCCGTTAGAAAATGT | 16 |
| | F: CAACCATTAGAAAATGTCTTTAT | 65 |
| RSV B | R: XGTTTTACCAATCGACATGT | 20 |
| | F: ATTGCATTTGGTTTCTTTTA | 19 |
| PIV 1 | F: TGAGTGATTAAGTTTGATGA | 25 |
| | R: XATTATTACCIGGACCAAG | 66 |
| PIV 2 | F: GGGTTGATTGTGGCCCA | 28 |
| | R: XCTGAGACTTGCTTTCTATTATTATAATGATAC | 67 |
| PIV 3 | F: ATGCTTATACCTCAAATCTA | 68 |
| | F: ATGCTTATACCTCGAATCTA | 69 |
| | R: XTAGGATTTAAGTCAGGTACC | 70 |
| | R: XTGGGATTTAAGTCAGGTACC | 71 |
| PIV 4A | F: GGGCGATTTCAATTTTT | 34 |
| | R :XGCAGAGGGTCGATTATATA | 35 |
| PIV 4B | F: TGTGCAGGTGCTTTC | 37 |
| | R: XCCCATAAGGCAAGAAG | 38 |
| HRV | F: AGCCTGCGTGGC | 1 |
| | R: XCGGICACCCAAAGTAGT | 72 |
| AdV B | F: CAGTACTCTGTTGTCGCC | 73 |
| | R: XGGGTCTGGTGCAGTT | 47 |
| AdV C | F: CTGAAGTACGTCTCGG | 49 |
| | R: XGCTACCCCTTCGATG | 50 |
| AdV E | F: CATTGGCATAGAGGAAGT | 52 |
| | R: XAGATGCAGGTTCTGAA | 53 |
| hMPV | F: GACCIATGGAATTCCCA | 74 |
| | R: XTGTTCTAC-I-ACACTCATTAT | 75 |
| CoVOC43 | F: TGATCAAATTGCTAGTCTTG | 7 |
| | R: XCTTATTCAAAATTTTCTGTCTG | 8 |
| CoVNL63 | F: TGGCTTTAAAGAACTTAGG | 13 |
| | R: XGAAAGAGGCTTATTAGGTT | 76 |
| CoV229E | F: ATTTCATGCTTTTGTTCTT | 10 |
| | R: XATAAAAAGTCAGCGAAAAC | 11 |

I = Deoxy-Inosine, X = Deoxy-isoC

TABLE 8
Sequences of RMA TSE Primers

| Viral | | |
|---|---|---|
| targets | TSE Primer | SEQ ID NO: |
| MHV | YYGGYATG-C3-CATAGCAGCCCGAGGATCT | 77 |
| HRV | YCYGAAGY-C3-CCGGC-I-CCTGAATG-I-GGCTAA | 78 |
| | YCYGAAGY-C3-GCCCCTGAATGCGGCTAA | 79 |
| CoVOC43 | GGAGYTCY-C3-AGGATGCCACTAAACCTCAGC | 80 |
| CoVNL63 | CCTGYGTY-C3-CAGTCGAAGTCACCTAGTTCTTCTG | 81 |
| CoV229E | CTYGYYTG-C3-ACTCTTGGCAGAAGTTTGAGAAGA | 82 |
| hMPV | CTYCYYAT-C3-GT-I-TTCCAAAATGCAATCAGCTG | 83 |
| AdVB | GCYCTCGY-C3-GTCAACGGGCACAAAGCGCA | 84 |
| AdVC | YYGCAYGC-C3-GGCGTCCTGGCCCGAG | 85 |
| AdVE | YCCYGYAG-C3-AAGGATTGCCTACATGGATTTCATTAGC | 86 |
| PIV4A | YCCYGYAG-C3-AAGGATTGCCTACATGGATTTCATTAGC | 87 |
| PIV4B | AYYGACGY-C3- | 88 |
| | CAATGATCTTTTATTTTCGCAATTATGTTTGTTT | |
| InfA1 | TYCGTGYY-C3-AATGGCTAAAGACAAGACCAATCCTGTCA | 89 |
| InfA2 | TYCGTGYY-C3-GAGCTGGCCAIAACCATTCTGTTC | 90 |
| InfB | AYCAGCYT-C3-GGACGTCTTCTCCTTTTCCCATTCCAT | 91 |
| RSVA | TYATCYCG-C3-GTTTTGCCATAGCATGACACAATG | 92 |
| RSVB | AGYCGGAY-C3- | 93 |
| | CTATTGTTATGACACTGGTATACCAACCTGTTC | |
| | AGYCGGAY-C3- | 94 |
| | AATTCTATTGTTATGACACTGGTATACCAACCTG | |
| PIV3 | CYYGYGAT-C3-ACTCGAGGTTGTCAGGATATAGGAA | 95 |
| | CYYGYGAT-C3-ACTCGAGGTTGCCAGGATATAGGAA | 96 |
| PIV1 | YCACYACG-C3-TGCATCACCAATTGATAATGAAGGT | 97 |
| PIV2 | YCAAYYCC-C3- | 98 |
| | TGCCCTGTTGTGTTTGGAAGAGATATGACTC | |

I = Deoxy-Inosine, Y = Deoxy-isoG, C3 = C3 aliphatic linker

With the primers, 155 samples of the original 689 were retested. The samples chosen for retesting are the following: all discordant samples, five concordant negatives, 24 concordant positives and six samples that RMA determined to be positive for more than one virus. Of the concordant positives retested, 6 were InfVA, 6 were InfVB, 2 RSV A and 2 RSV B. Of all 155 retested samples, the RMA system continued to report 30 discordants. Discordants included 24 RMA positives and 5 RMA negatives.

When this new data was used to replace the initial data for the 155 retested samples, the overall sensitivity and specificity numbers increased from 82% to 99% and 84% to 87% respectively. In particular, of the 145 standard algorithm positive InfB samples all but 1 was correctly identified by the RMA. This translates into 98% sensitivity and 100% specificity with confidence intervals of 4% and 1% respectively for the RMA InfVB analysis. The assay results also reported high sensitivity and specificity percentages for the remaining targets. Yet with the exception of Flu A and B, sensitivity confidence intervals were typically high due to the lack of positive samples in the study.

The RMA positive samples for targets which current WSLH culture methods do not detect (9 NL63 and 5 human metapneumovirus positive samples) were tested by single target real time RT-PCR assays. Real time PCR data showed all but one (hMPV RT-PCR negative sample) to be concordant with the RMA results.

In sum, the standard testing algorithm commonly performed at the State Laboratory detected 504 (73%) positive samples and 185 target negatives for the 689 samples tested. Analyzing those same 689 samples, the RMA system detected 529 (77%) positives for one or more targets and 160 complete target negatives. There were 29 discordant calls between the two systems. Fourteen samples reported positive results for targets not tested by the standard State Laboratory algorithm of which 13 were confirmed by real-time PCR. When results using our standard algorithm were considered “true positives,” the RMA showed an overall sensitivity of 99% and overall specificity of 87%. In total, the RMA detected an additional 38 viruses of which 10 were mixed infections.

### Example 11

### Development of hMPV Fusion Gene Primers

Additional primer and probes designs were tested in the detection of hMPV. Primers and probes used in the following example are as shown in Table 9.

TABLE 9
Sequences of hMPV Fusion Gene Primers

| | | SEQ |
|---|---|---|
| | | ID |
| Name | Sequence | NO: |
| DM1217 | XCATGGTGCAGCTGCCGATCTTTGG | 236 |
| DM1218 | XTACATGGTGCAGCTGCCGATCTTTG | 237 |
| DM1219 | XAATTTACATGGTGCAGCTGCCAA | 238 |
| DM1220 | XGATTTACATGGTTCAATTGCCGA | 239 |
| DM1221 | CTYCYYAT-C3-ACAGTTGACCCTGCATTCTGACAATACCA | 240 |
| DM1222 | CTYCYYAT-C3-ACAGTGGATCCTGCATTTTTACAATACCA | 241 |
| DM1223 | CTYCYYAT-C3-GGGGCTGCTTTTACTATCCAGCAAG | 242 |
| DM1224 | CTYCYYAT-C3-GGAGCTGCCTTGATTATCCAACAAG | 243 |
| DM1225 | CTYCYYAT-C3-GGAGCTGCTTTGATTATCCAACAAG | 244 |
| DM1226 | TTGGGTAGTAAACAGTTGACCC | 245 |
| DM1227 | TTGGGTAGTAAACAGTGGATCC | 246 |
| DM1228 | TTTGGGTAGTAAACAGTGGATCCTGC | 247 |
| DM1229 | TTGGGTAGTAAACAGTGGATCCTGCA | 248 |
| DM1230 | TTGGGTAGTAAACAGTAGATCCTGCAT | 249 |
| DM1250 | CTYCYYAT-C3-GGGGCTGCTTTTACTATCCAGCAGG | 250 |

The reaction was performed as described in Example 5 above. The primers were tested using a diluted plasmid clone as the target nucleic acid. Dilutions were made at 5000, 500, and 50 copies per reaction. PCR and PLx primers were supplied at 200 nM each and the TSE primers were supplied at 15 nM. Combinations of the above PLx/TSE/PCR primers were selected for the detection of hMPV are shown in Table 10.

TABLE 10
Combinations of hMPV Fusion Gene Primers

| | PCR | | |
|---|---|---|---|
| | (Forward) | PLx (Reverse) | |
| | Primer | Primer | TSE |
| Group 1 | DM1226/ | DM1217 | DM1221/DM1222 |
| | DM1227 | | |
| Group 2 | DM1228 | DM1218 | DM1222/DM1224/DM1250 |
| Group 3 | DM1229 | DM1217/DM1220 | DM1221/DM1222/DM1250 |
| Group 4 | DM1230 | DM1218/DM1220 | |

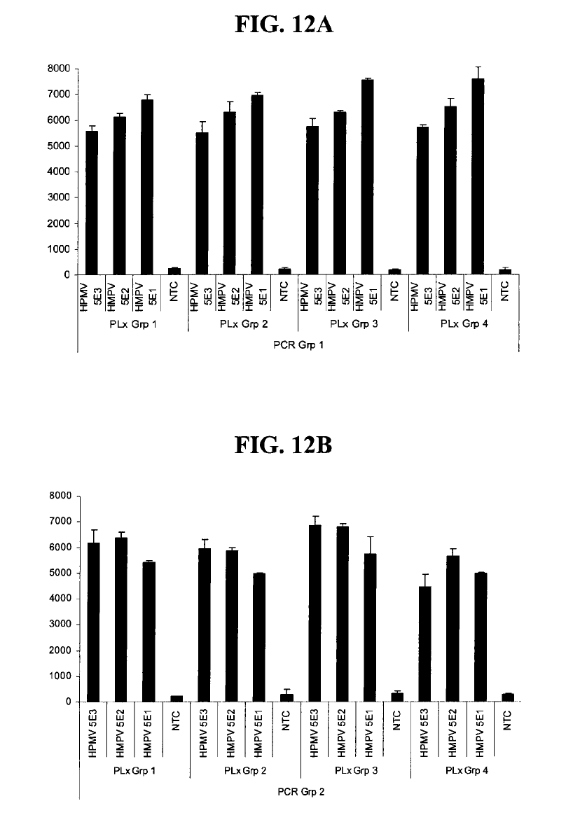

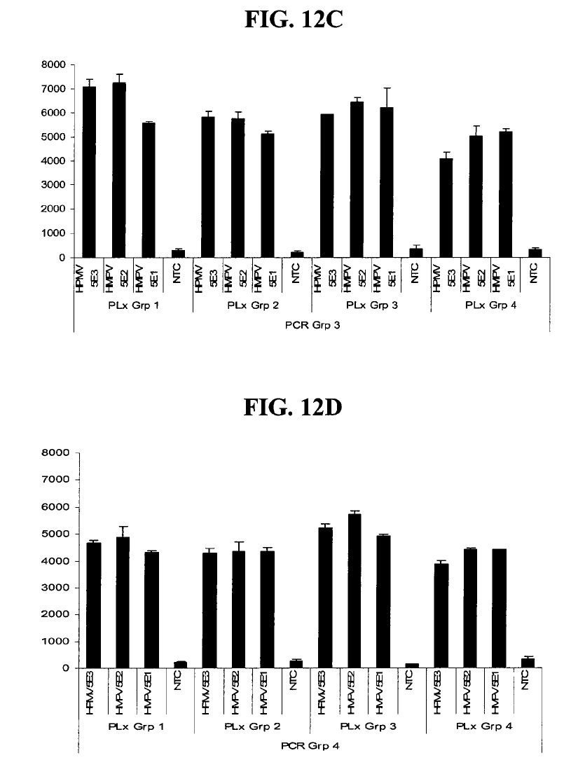

Results were considered positive when MFI output was at least 6 standard deviations over the average background. The results for Groups 1 to 4 are shown in FIGS. 12A to 12D, respectively. The data show that each of the combinations of PCR primers and TSE primers were capable of detecting mHPV with as few as 50 copies per reaction.

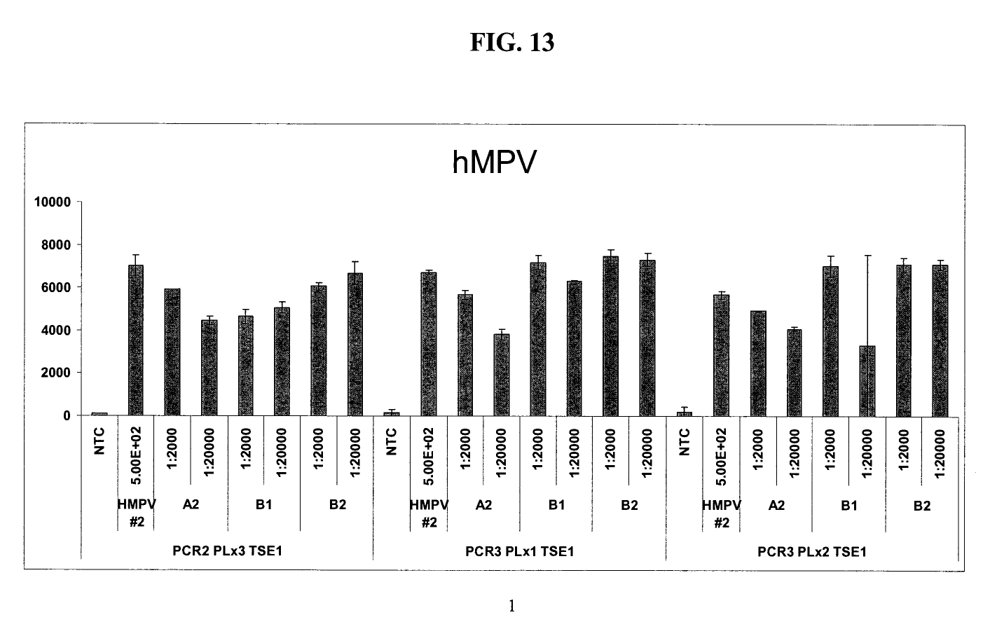

In another experiment, primer and probe combinations were tested against three serotypes of hMPV (A2, B1, and B2). The results are shown in FIG. 13. Three combinations were tested. The first combination was the PCR primer from Group 2 (DM1228), the PLx primer from Group 3 (DM1217/DM122), and the TSE probe from Group 1 (DM1221/DM1222). The second combination was the PCR primer from Group 3 (DM1217/DM1220), the PLx primer from Group 2 (DM1218), and the TSE probe from Group 1 (DM1221/DM1222). The third combination was the PCR primer from Group 3 (DM1217/DM1220), the PLx primer from Group 1 (DM1217), and the TSE probe from Group 1 (DM1221/DM1222). The results are shown in FIG. 13 and show that each of the combinations of PCR primers and TSE primers were capable of detecting hMPV serotypes A2, B1, and B2.

It will be readily apparent to one skilled in the art that varying substitutions and modifications may be made to the invention disclosed herein without departing from the scope and spirit of the invention. The invention illustratively described herein suitably may be practiced in the absence of any element or elements, limitation or limitations which is not specifically disclosed herein. The terms and expressions which have been employed are used as terms of description and not of limitation, and there is no intention that in the use of such terms and expressions of excluding any equivalents of the features shown and described or portions thereof, but it is recognized that various modifications are possible within the scope of the invention. Thus, it should be understood that although the present invention has been illustrated by specific embodiments and optional features, modification and/or variation of the concepts herein disclosed may be resorted to by those skilled in the art, and that such modifications and variations are considered to be within the scope of this invention.

In addition, where features or aspects of the invention are described in terms of Markush groups or other grouping of alternatives, those skilled in the art will recognize that the invention is also thereby described in terms of any individual member or subgroup of members of the Markush group or other group.

Also, unless indicated to the contrary, where various numerical values are provided for embodiments, additional embodiments are described by taking any 2 different values as the endpoints of a range. Such ranges are also within the scope of the described invention.

All references, patents, and/or applications cited in the specification are incorporated by reference in their entireties, including any tables and figures, to the same extent as if each reference had been incorporated by reference in its entirety individually. Definitions that are contained in incorporated text are excluded to the extent they contradict definitions in this disclosure.

What is claimed is:

1. A method of detecting a member of a human respiratory viral group in a sample, the method comprising:
(a) reacting the sample and a reaction mixture to obtain an amplicon, the reaction mixture comprising a set of primer pairs specific for each viral group to be detected, and wherein at least one primer of each pair comprises at least one non-standard nucleobase selected from iso-G and iso-C;
(b) hybridizing a target specific extension primer to the amplicon, wherein the target specific extension primer is different than any primer of the primer pairs, and wherein the target specific extension primer comprises a tagging sequence that comprises at least one non-standard nucleobase selected from iso-G and iso-C;
(c) extending the hybridized target specific extension primer in the presence of a labeled nucleotide to obtain a labeled target oligonucleotide;
(d) hybridizing the labeled target oligonucleotide to an immobilized oligonucleotide that hybridizes to the tagging sequence;
(e) detecting the labeled target sequence.

2. The method of claim 1, wherein the human respiratory viral groups comprise two or more members selected from the group consisting of rhinovirus, respiratory syncytial virus, parainfluenza virus, influenza virus, metapneumovirus, adenovirus, coronavirus, bocavirus, and enterovirus.

3. The method of claim 1, wherein the human respiratory viral groups comprise two or more members selected from the group consisting of HRV, EnV, RSVA, RSVB, MPV, PIV1, PIV2, PIV3, PIV4a, PIV4b, InfVA, InfVB, CoVOC43, CoV229E, CoVNL63, AdVB, AdVC, and AdVE.

4. The method of claim 1, wherein the human respiratory viral groups comprise two or more members selected from the group consisting of EnV, CoVOC43, CoV229E, CoVNL63, AdVB, AdVC and AdVE.

5. The method of claim 1, wherein the human respiratory viral groups comprise two or more members selected from the group consisting of: EnV, CoVOC43, CoV229E, CoVNL63, AdVB, AdVC, AdVE, HRV, RSVA, RSVB, MPV, PIV1, PIV2, PIV3, PIV4a, PIV4b, InfVA, and InfVB.

6. The method of claim 1, wherein the target specific extension primer includes a linker sequence.

7. The method of claim 1, wherein the immobilized oligonucleotide is coupled to a solid support.

8. The method of claim 7, wherein the solid support comprises microspheres.

9. The method of claim 8, wherein the step of detecting comprises flow cytometry.

10. The method of claim 1, wherein step (a) is performed in the presence of one or more of the following: iso-C and iso-G nucleotide triphosphates.

11. The method of claim 1, wherein the labeled nucleotide of step (c) comprises one or more of the following: iso-G and iso-C nucleotide triphosphate coupled to a detectable label.

12. The method of embodiment 11, wherein the label comprises biotin.

13. The method of claim 12, wherein detection comprises contacting fluorescent strepavidin-phycoerythrin with the biotin label.

14. The method of claim 1, wherein the immobilized oligonucleotide includes a non-standard nucleobase, and wherein the non-standard nucleobase of the tagging sequence hybridizes to the non-standard nucleobase of the immobilized oligonucleotide.

15. The method of claim 1, wherein the primer pairs specific for each viral group are selected from one or more primer pairs of the group consisting of: SEQ ID NO:1-2, SEQ ID NO: 4-5, SEQ ID NO: 7-8, SEQ ID NO: 10-11, SEQ ID NO: 13-14, SEQ ID NO: 16-17, SEQ ID NO: 19-20, SEQ ID NO: 22-23, SEQ ID NO: 25-26, SEQ ID NO: 28-29, SEQ ID NO: 31-32, SEQ ID NO: 34-35, SEQ ID NO: 37-38, SEQ ID NO: 40-41, SEQ ID NO: 43-44, SEQ ID NO:46-47, SEQ ID NO: 49-50, and SEQ ID NO: 52-53.

16. The method of claim 15, wherein the target specific extension primer for each viral group is selected from the group consisting of: SEQ ID NO: 3, 6, 9, 12, 15, 18, 21, 24, 27, 30, 33, 36, 39, 42, 45, 48, 51, and 54.

17. The method of claim 1, wherein the primer pairs specific for each viral group are selected from one or more primer pairs of the group consisting of: SEQ ID NO: 58-59, SEQ ID NO: 60-62, SEQ ID NO: 61-62, SEQ ID NO: 63-64, SEQ ID NO: 16-17, SEQ ID NO: 17-65, SEQ ID NO: 19-20, SEQ ID NO: 25-66, SEQ ID NO: 28-67, SEQ ID NO: 68-70, SEQ ID NO: 69-70, SEQ ID NO: 69-71, SEQ ID NO: 34-35, SEQ ID NO: 37-38, SEQ ID NO: 1-72, SEQ ID NO: 73-47, SEQ ID NO: 49-50, SEQ ID NO: 52-53, SEQ ID NO: 74-75, SEQ ID NO: 7-8, SEQ ID NO: 13-76, and SEQ ID NO: 10-11.

18. The method of claim 17, wherein the target specific extension primer for each viral group is selected from the group consisting of: SEQ ID NO: 77-98.
